(19) United States
(12) Patent Application Publication
(10) Pub. No.: US 2025/0332407 A1
(43) Pub. Date: Oct. 30, 2025

(54) TRANSVASCULAR BRAIN STIMULATION

(71) Applicants: Michael HAINES, Ormond (AU); Gil Simon RIND, Northcote (AU); Rahul SHARMA, Dover, DE (US); Brian B. MARTIN, Dover, DE (US)

(72) Inventors: Michael HAINES, Ormond (AU); Gil Simon RIND, Northcote (AU); Rahul SHARMA, Dover, DE (US); Brian B. MARTIN, Dover, DE (US)

(21) Appl. No.: 19/195,568

(22) Filed: Apr. 30, 2025

Related U.S. Application Data
(60) Provisional application No. 63/640,723, filed on Apr. 30, 2024.
(60) Provisional application No. 63/671,922, filed on Jul. 16, 2024.
(60) Provisional application No. 63/745,728, filed on Jan. 15, 2025.
(60) Provisional application No. 63/765,007, filed on Feb. 28, 2025.

(51) Int. Cl. A61N 1/05; A61B 17/00; A61B 17/34

(52) U.S. Cl. CPC A61N 1/0534; A61B 17/3468; A61B 2017/00026; A61B 2017/00557

(57) ABSTRACT

Disclosed herein are methods and devices for transvascular placement of electrodes on a surface of a brain or in deep brain structures for the purpose of neuromodulation. The device can comprise a delivery catheter comprising a lumen. The device can comprise a piercing assembly extending through the delivery catheter, wherein the piercing assembly comprises a piercing assembly catheter and a needle. The device can comprise one or more electrodes configured to contact the brain tissue; and wherein the piercing assembly catheter comprises an opening in a wall thereof such that when the delivery catheter and the piercing assembly catheter are positioned within a vessel, the needle extends through the opening to puncture a vessel wall.

### CROSS-REFERENCE TO RELATED APPLICATION

This application claims the benefit of U.S. Provisional Application No. 63/640,723, filed Apr. 30, 2024, U.S. Provisional Application No. 63/671,922, filed Jul. 16, 2024, U.S. Provisional Application No. 63/745,728, filed Jan. 15, 2025, and U.S. Provisional Application No. 63/765,007, filed Feb. 28, 2025, the content of each are incorporated herein by reference in their entireties.

### TECHNICAL FIELD OF THE INVENTION

The present invention relates to a medical device for accessing regions or surfaces of the brain, specifically for implantation of electrodes and/or neural sensing/stimulation devices.

### BACKGROUND OF THE INVENTION

Presently, conventional approaches exist that attempt to access regions of the brain for stimulation of neural tissue or detecting neural signals. Such approaches that are generally known include deep brain stimulation (“DBS”), which involves implanting electrodes within certain areas of a brain where the electrodes produce electrical impulses in an attempt to stimulate or regulate brain activity for a therapeutic or other purpose, as well as electrocorticography (“ECoG”), which enables neuromonitoring of brain regions for a diagnostic purpose and/or for the purpose of a brain computer interface system.

Typically, implantation of such neural devices involves creating burr holes in the skull to implant electrodes and surgery to implant a controller or pacemaker-like device that is electrically coupled to the electrodes to control the stimulation or to sense neural signals. This device can be positioned under the skin in the chest. The amount of stimulation in deep brain stimulation can be controlled by the controller or pacemaker-like device where a wire/lead connects the controller device to electrodes positioned in the brain.

DBS can be used to treat a number of neurological conditions, such as tremors, Parkinson's disease, dystonia, epilepsy, Tourette syndrome, chronic pain, and obsessive-compulsive disorder. In addition, DBS has the potential for the treatment of major depression, stroke recovery, addiction, and dementia. Moreover, implanting electrodes in neural tissue can influence the efficacy of stimulating and/or recording neural tissue (e.g., using brain-computer interfaces), such as decoding thoughts from neural signals.

The positioning of electrodes on the brain or into neural tissue can present risks, especially when using a transcranial approach. FIG. 1 illustrates a conventional transcranial approach of accessing regions of the brain 12 of an individual 10 with a brain stimulation/monitoring device 20, usually including an electrode carrier 22, having a plurality of electrodes 24 that are implanted within a target region 30 of the brain 12. As shown, the implantation requires surgical penetration, e.g., a craniotomy, of the skull 14 such that the electrodes 24 are directed towards an area of interest 30. In addition, the device 20 includes a lead 28 that couples the electrodes 24 to a controller/transceiver/generator 26. The lead 28 and controller 26 can be surgically implanted within the individual 10 or positioned on an exterior surface of the individual 10.

There are a number of risks associated with the general surgery required to surgically implant the device 20 in conventional DBS procedures. Furthermore, there are risks in the process of the DBS procedure itself, given that conventional procedures require an approximation or non-invasive attempt to locate the region of interest 30. Then, the physician must attempt to physically position the electrodes 22 of the device 20 in or near the area of interest 30 so that the desired effect can be achieved. In certain cases, the positioning of the electrodes 20 can be a trial-and-error approach requiring multiple surgical attempts and multiple surgical insertion sites. Regardless of the number of attempts, the act of inserting the device 20 to position the electrodes 22 in the area of interest 30 creates collateral damage to brain tissue located in the path between the area of interest and the insertion point in the cranium.

Currently, the surgical risks involved in such procedures can include bleeding in the brain, stroke, infection, collateral damage to brain tissue, collateral damage to vascular structures in the brain, temporary pain, and inflammation at the surgical site.

However, the conventional approaches intended to access the many subnetworks of the brain are deficient such that the conventional approaches are unable to maximize the benefit of accessing and directly communicating/stimulating these subnetworks. For example, in the case of using a brain stimulation device 20 to treat Parkinson's disease, an electrode 24 or electrode carrier 22 must be positioned through a significant amount of brain structures to ensure the electrode 24 is positioned at or near a target site 30. Once positioned, either the lead 28 or the electrode carrier 22 comes out through the skull 14 under skin and then is positioned to reach the controller 26, which is typically positioned on or in the chest.

Neurovascular electrophysiology and therapeutic devices are limited in their positioning over or within the cortex by the highly variable physical presence and pathway that veins take. Therefore, to gain access to wider regions of functionally rich brain regions for recording and stimulation purposes, the ability to deploy recording and stimulation arrays without the spatial limitations of the vascular network will prove highly valuable.

There remains a need for implantation of electrodes and/or neural sensing/stimulation devices while minimizing collateral damage to tissue from the procedure. There especially remains a need for a transvascular approach to create a location or space within the dura matter so that a vascular approach can deliver electrodes or other devices to the space. There also remains a need for deploying electrode steering devices to locations adjacent to or in brain tissue and closing vessel punctures post-delivery.

It is noted that the devices discussed herein can allow transvascular placement to position electrodes in deep brain structures for the purpose of neuromodulation, including movement disorders, epilepsy, and depression. The electrodes can reside in a deep brain region in an intraparenchymal location with a penetrating electrode array. Alternatively, the electrodes can be surface electrodes. These devices are able to sense and/or stimulate the brain region to reduce a particular symptom (e.g., tremor in Parkinson's or seizures in epilepsy in the case of stimulation). The devices can be open-loop or closed-loop. In addition, the electrode devices can perform intracranial electroencephalography such as ECoG, for neuromonitoring of brain regions and/or brain computer interface systems.

The following U.S. patents describe the use of the venous network to access brain tissue in order to form a shut to relieve cranial pressure: U.S. Pat. No. 9,387,311 issued on Jul. 12, 2016, U.S. Pat. No. 9,545,505 issued on Jan. 17, 2017, U.S. Pat. No. 9,662,479 issued on May 30, 2017, U.S. Pat. No. 9,669,195 issued on Jun. 6, 2017, U.S. Pat. No. 10,272,230 issued on Apr. 30, 2019, U.S. Pat. No. 9,724,501 issued on Aug. 8, 2017, U.S. Pat. No. 10,279,154 issued on May 7, 2019, U.S. Pat. No. 10,058,686 issued on Aug. 28, 2018, U.S. Pat. No. 10,758,718 issued on Sep. 1, 2020, U.S. Pat. No. 10,765,846 issued on Sep. 8, 2020, U.S. Pat. No. 10,307,576 issued on Jun. 4, 2019, U.S. Pat. No. 10,307,577 issued on Jun. 4, 2019, U.S. Pat. No. 11,013,900 issued on May 25, 2021, U.S. Pat. No. 11,633,578 issued on Apr. 25, 2023, the entirety of which is incorporated by reference. The present disclosure can incorporate such access and provides novel methods, devices, and systems for locating, directing, and/or implanting neural sensing/stimulation devices within deep brain tissue.

### SUMMARY OF THE INVENTION

The present disclosure includes methods, devices, and systems that enable deposition of electrodes and other recording devices in information-rich areas of the brain or the deposition of open or closed-loop feedback implantable brain stimulator via the venous system of the brain.

An example of such a system can include multiple elements that permit venous access via a catheter that delivers a guide catheter from the jugular vein and punctures into the inferior petrosal sinus.

Variations of the present disclosure include systems for accessing a target region of a brain from a vessel. For example, such a system can include a catheter body having a distal region; a navigation device slidably advanceable through the catheter body to the distal region, the navigation device including a distal portion that is configured to be steerable independently of the catheter body and an expandable member at the distal portion, where the expandable member is configured to anchor the distal portion exterior to the vessel; a guidewire configured to extend through a working lumen of the navigation device; and an electrode carrier configured to be advanced through the working lumen of the navigation device and through the expandable member such that the electrode carrier can be advance in a straight line from an opening in the expandable member to the target region of the brain.

Variations of the present disclosure can also include a first expandable structure located at the distal region of the catheter body and configured to bias the catheter body against a wall of the vessel.

The systems described herein can include a catheter body that includes a passage exiting a side opening in a sidewall at the distal region, wherein the passage is configured such that advancement of the navigation device therethrough causes the navigation device to exit the catheter body at the side opening.

Variations of the present disclosure can include systems that further include a bone-penetrating structure configured for sliding through the catheter body.

The electrode carrier described herein can include a multitude of electrode configurations, such as, a linear electrode array, an electrode array having a planar electrode region configured to have a delivery profile (i.e., a low profile suitable for delivery through a catheter) and expandable to a planar or deployment profile when advanced out of the navigation device. The array can include a planar electrode region includes a foldable structure such that expansion of the planar electrode region from the delivery profile to the planar profile includes unfolding the foldable structure; and/or an array with a planar electrode region that includes an expandable structure such that expansion of the planar electrode region from the delivery profile to the planar profile includes expanding the expandable structure to expose one or more electrodes.

Variations of the present disclosure include a system having a grommet structure configured for placement within an opening in a wall of the vessel, where the grommet structure allows passage of the catheter body or navigation device therethrough.

The present disclosure can include a system having a stent structure having at least one opening in a side of the stent structure for passage of the catheter body or navigation device therethrough when positioned in the vessel.

The stents disclosed herein can include a stent body expandable from a deployment configuration to an expanded configuration; a port extending from a side of the stent body, the port having a passage and having a sharp edge on a free end of the port opposite to the stent body; a polymer covering the port and the sharp edge, wherein the polymer is configured to dissolve or degrade over a period of time, wherein when deployed in a vessel the stent body biases the polymer covering the sharp edge against a wall of the vessel, wherein after the polymer dissolves or degrades, the stent body urges the sharp edge of the port into the wall of the vessel such that the wall of the vessel adheres to a portion of the port to secure the port in place.

The present disclosure also includes methods of transvascular access to a region of a brain. For example, such methods can include advancing a catheter into a vessel; anchoring the catheter within the vessel; passing the catheter through a vessel opening in a wall of the vessel and adjacent to brain tissue; deploying a navigation device from the catheter to a location exterior to the vessel; expanding an expandable structure located at a distal portion of the catheter, where the expandable structure anchors to a location exterior to the vessel; steering the expandable structure to align a travel path from an opening of the expandable structure to a target region; and advancing an electrode carrier from the opening of the expandable structure along the travel path and to the target region.

The methods described herein can include an electrode carrier that is advanced over a surface of the brain. Alternatively, or in combination, the methods can include advancing the electrode carrier from the opening of the expandable structure along the travel path and to the target region includes advancing the electrode carrier through a tissue of the brain.

Variations of the present disclosure include a method for expanding an electrode carrier in a planar direction over the target region.

Variations of the present disclosure include a method wherein the electrode carrier is configured to form a two-dimensional or three-dimensional array when expanded.

Variations of the present disclosure include a method wherein expanding the electrode carrier in the planar direction includes unfolding the electrode carrier from a folded state.

The methods described herein can include a catheter with a biasing portion of the catheter that urges the catheter against a wall of the vessel.

Additional variations of the present disclosure include methods for transvascular access to a region of a brain. Such methods can include advancing a catheter into a vessel; anchoring the catheter within the vessel; passing the catheter through a vessel opening in a wall of the vessel and adjacent to brain tissue; deploying a navigation device from the catheter to a location exterior to the vessel; expanding an expandable structure located at a distal portion of the catheter, where the expandable structure anchors to the exterior of the vessel; steering the expandable structure to align a travel path from an opening of the expandable structure to a target region; and advancing an electrode carrier from the opening of the expandable structure along the travel path and to the extravascular target region.

Additional variations of the present disclosure can include advancing a catheter into a vessel where a distal portion of the catheter includes at least one lumen terminating in a side opening in a sidewall of the catheter; anchoring the catheter within the vessel advancing a puncture catheter through the side opening of the catheter and through a wall of the vessel to create a vessel opening in the wall of the vessel; deploying an intermediate catheter over the puncture catheter into the vessel opening adjacent brain tissue; expanding one or more anchor members on the intermediate catheter to secure the intermediate catheter in place while extending through the vessel opening; removing the puncture catheter; advancing an electrode carrier through the intermediate catheter and towards the region of the brain; removing the intermediate catheter; delivering a substance from the catheter to seal a portion of the electrode carrier within the vessel opening; and removing the catheter such that the electrode carrier is positioned transvascularly within the brain. Expanding an expandable structure located at a distal portion of the catheter, where the expandable structure anchors to an exterior of the vessel; steering the expandable structure to align a travel path from an opening of the expandable structure to a target region; and advancing an electrode carrier from the opening of the expandable structure along the travel path and to the target region.

In another variation, the methods can include delivering a needle from the guide catheter that punctures the wall of the venous sinus (e.g., inferior petrosal sinus) and skull to enter the brain tissue and then delivering a steerable navigational device from the exterior of the vessel through a wall of the skull and into the brain. The device can include one or more anchors that anchor the catheter into position to permit targeted deployment of an electrode lead into the brain.

In another variation, the method can include manipulating the navigational device such that it can be repositioned in a three-dimensional space to precisely target a straight-line trajectory for the entry of the lead into the brain. The position of the anchor would manipulate the position of the catheter in relation to the entry position with relation to the brain, including:

The navigation device can include any number of sensors or markers that allow for non-invasive imaging to confirm positioning of the electrodes. Alternatively, or in combination, confirming the position of the anchor in 3D space can occur with a 2-way communication of an external stereotactic navigation system.

In another variation, the system can use an external magnetic system for manipulation of the navigation device.

Targets include all known deep brain stimulation targets. One example is the subthalamic nucleus, which can treat tremors associated with Parkinson's disease (which can be 20 mm away from the inferior petrosal sinus).

The present disclosure can be used in addition to the devices disclosed in the following patents/publications or in combination with aspects and features of the related disclosure of these patents, publications, and applications: U.S. Pat. No. 10,575,783 issued on Mar. 3, 2020, U.S. Pat. No. 10,485,968 issued on Nov. 26, 2019, U.S. Pat. No. 10,729,530 issued on Aug. 4, 2020, US20190336748 published on Nov. 7, 2019, US20200016396 published on Jan. 16, 2020, US20220253024 published on Aug. 11, 2022, U.S. Pat. No. 11,550,391 issued on Jan. 10, 2023, U.S. Pat. No. 11,672,986 issued on Jun. 13, 2023, US20220369994 published on Nov. 24, 2022, and U.S. Pat. No. 11,630,517 issued on Apr. 18, 2023, and U.S. application Ser. No. 18/792,965 filed on Aug. 2, 2024. The entirety of each of these is incorporated by reference.

### BRIEF DESCRIPTION OF THE DRAWINGS

FIG. 1 illustrates a conventional transcranial approach of accessing regions of the brain of an individual with a brain stimulation/monitoring device.

FIG. 2 represents the various layers of the human head, including a scalp, which overlies the periosteum lining the cranium/skull.

FIG. 3A illustrates an access device advanced into a jugular vein to permit navigation of a catheter within an inferior petrosal sinus.

FIG. 3B shows a directing structure advanced from a catheter.

FIG. 4A illustrates a catheter advancing into vessels within the brain of an individual 10 through the jugular vein.

FIG. 4B shows a magnified region of FIG. 4A to better illustrate the catheter advancing in general alignment with an axis of the catheter where the walls of the vessel run approximately parallel to the axis.

FIG. 5A illustrates a variation of a navigation device with a second anchor and illustrates an intended travel path from the navigation device.

FIG. 5B illustrates the navigation device of FIG. 5A being manipulated to alter the intended travel path to coincide with a region of interest.

FIG. 6A shows a variation of a navigation device to position an electrode carrier along an alignment path to place electrodes within or near a region of interest.

FIG. 6B illustrates a state where the catheter, navigation device, anchor, and other delivery elements are removed from the site to leave the electrodes positioned within the region of interest.

FIGS. 7A to 7F show another variation of a transvascular approach to position an electrode carrier within or near a region of interest.

FIG. 7G shows a partial cross-sectional view of an electrode carrier having a magnetic stylet extending within the carrier.

FIG. 8 illustrates implanted electrodes positioned within a region of interest in the brain and where a lead or electrode carrier extends partially through the vasculature towards a controller.

FIGS. 9A to 9F illustrate examples of a process of accessing the subarachnoid cavity or other deep brain region.

FIG. 10 illustrates one variation of an assembly having a guide catheter with a sideport having a ramp to track a puncture/microcatheter assembly through a wall of the vessel.

FIG. 11A shows another example of advancing a catheter within a vessel where a secondary piercing assembly is advanced within the vessel in a collapsed state.

FIG. 11B illustrates deployment of the piercing assembly from the guide catheter from the collapsed state to a deployed state.

FIG. 11C illustrates a variation of a piercing assembly similar to the one shown in FIG. 11B.

FIGS. 11D and 11E show additional variations of a puncture assembly positioned distally to a catheter within walls of a vessel.

FIG. 11F is a cross-sectional view to show a variation of a piercing assembly having an interlocking structure with the interior of the guide catheter.

FIGS. 11G and 11H illustrate another variation of a locking balloon that locks two catheters together.

FIG. 11I shows another example of a catheter positioned within a vessel to deploy a piercing assembly comprising a needle and microcatheter.

FIG. 11J illustrates a catheter having a rotational body that allows rotation or the application of torque within a vessel.

FIGS. 11K and 11L illustrate a catheter having different type of a rotational body that allows rotation or the application of torque through linear motion of a line to function as a cam follower assembly.

FIG. 12 shows another variation of a diversion device that creates a work region within the vessel.

FIGS. 13A and 13B illustrate an example of a controlled system for achieving a precise vessel puncture within the delicate neural anatomy that can be used with any of the devices described herein.

FIG. 14A is an illustration showing a state after a wall of the vessel is punctured.

FIG. 14B illustrates one variation of a catheter having forceps-type blunt jaws at the distal end.

FIG. 14C shows another variation where the device comprises a balloon catheter with a 2D “pancake” profile balloon.

FIG. 14D shows another variation where the device comprises a coaxial or axial balloon catheter having any number of balloons.

FIG. 14E illustrates an optional device that can be advanced adjacent to brain tissue to separate the dura and either create a pocket through blunt dissection or to create a path.

FIG. 14F illustrates an additional aspect of a method of creating a space or cavity within or adjacent to dural tissue.

FIGS. 15A to 15C illustrate another variation of an electrode device passing through an opening in a vessel.

FIGS. 16A to 16E illustrate another variation of an electrode device coupled to a balloon catheter that expands to deploy the electrode device and can then be removed leaving the electrode device in place.

FIG. 17A illustrates an additional concept of placement of electrodes or an electrode array at a desired region of tissue using improved control mechanisms.

FIG. 17B shows a catheter withdrawn from the electrode assembly, leaving the electrode assembly in place.

FIGS. 17C and 17D show another variation where a catheter is advanced from the vessel 6 to position a balloon over brain tissue and the electrode assembly is pulled in place using the balloon.

FIGS. 17E and 17F illustrate another example of pulling electrodes into place using a wire 194 and a double-balloon system.

FIGS. 18A and 18B show another variation of a balloon catheter that can be positioned between brain tissue and the dura and unrolled to deploy an electrode assembly.

FIG. 19 illustrates the use of a positioning catheter advanced through an opening in the skull to position a catheter or the electrode assembly.

FIGS. 20 and 21 illustrate additional systems for use in delivering the electrode devices to the desired target regions.

FIGS. 22A to 22C illustrate a stent structure having a port with a sharp distal edge transitioning to a medical textile brim section on the proximal side.

FIGS. 23A to 23D show various examples of stent scaffold-style 2D self-expanding arrays.

FIG. 24A illustrates a stent strut having one or more penetrating surface electrodes on the stent structure.

FIG. 24B illustrates a surface-mounted, flexible tine feature that carries one or more electrode surfaces.

FIGS. 25A and 25B show variations of systems where sensing/recording electrodes are separated or spaced from an anchoring structure.

FIGS. 26A and 26B illustrate a self-expanding or balloon-expanded stent frame with a large cell size with a region of thin, soft, self-healing, polymer material between cell(s).

FIG. 27A illustrates a variation of a guide catheter with an expanding stent-type feature that provides positional fixation within a target vessel.

FIG. 27B is a partial cross-sectional view of the device of FIG. 27A showing the internal lumen of the guide, including a lumen-reducing balloon.

FIG. 27C illustrates another variation of a deep brain stimulation burrowing guidewire that can optionally have a distal threaded portion that can be advanced into brain tissue and achieve penetration via rotation.

FIGS. 28A to 28C illustrate an optional design of a recording array for deployment through the vessel wall.

FIGS. 29A to 29C illustrate a folding array that interchanges between two deployment states.

FIGS. 30A and 30B illustrate an expandable electrode carrier extending out of a superior sagittal sinus for positioning over brain tissue.

FIG. 30C illustrates a guide catheter within a vessel, where the catheter includes expanding features to secure a side port against a wall of the vessel.

FIGS. 31A to 31D illustrate additional variations of devices that form planar electrode carriers.

FIG. 32A shows a flexible microarray comprising a polymer/ceramic substrate.

FIG. 32B shows the array of FIG. 32A positioned on cortical tissue.

FIGS. 33A and 33B illustrate additional variations of devices.

FIGS. 34A and 34B illustrate perspective and side views, respectively, of an example of an expanding vessel puncture port or grommet.

### DETAILED DESCRIPTION

The present methods and devices relate to electrodes directly accessing, monitoring, and/or communicating with specific regions of the brain via a vascular approach for the purpose of using the direct access to minimize damage to adjacent tissues within the brain and anatomy.

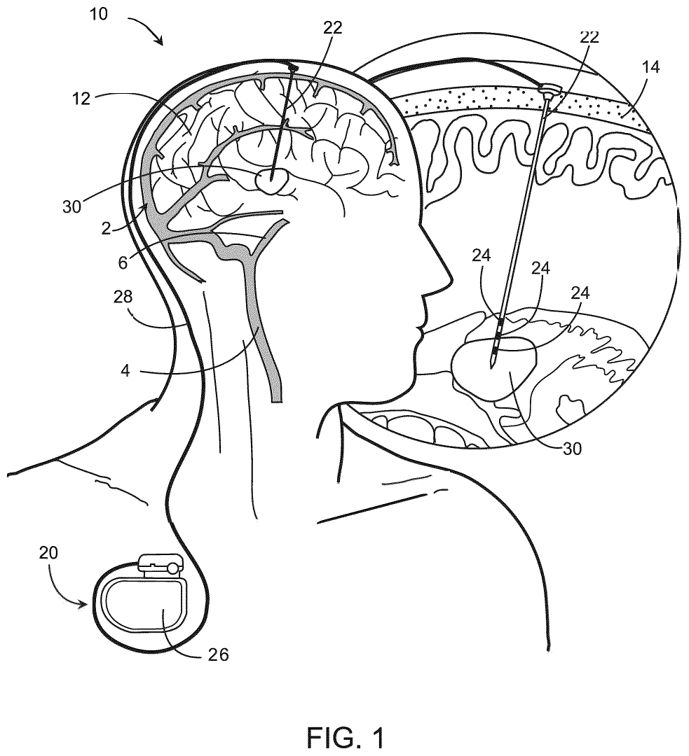

FIG. 1 illustrates a vascular network 2 of the brain extending from the jugular vein 4. The present disclosure uses the venous network 2 of the brain 12 to access target regions of interest for DBS. While the examples below discuss accessing a specific vessel 6, the inferior petrosal sinus (IPS) that branches from the jugular vein 4, any vessel located within the brain 12 can be used for access of neural tissue.

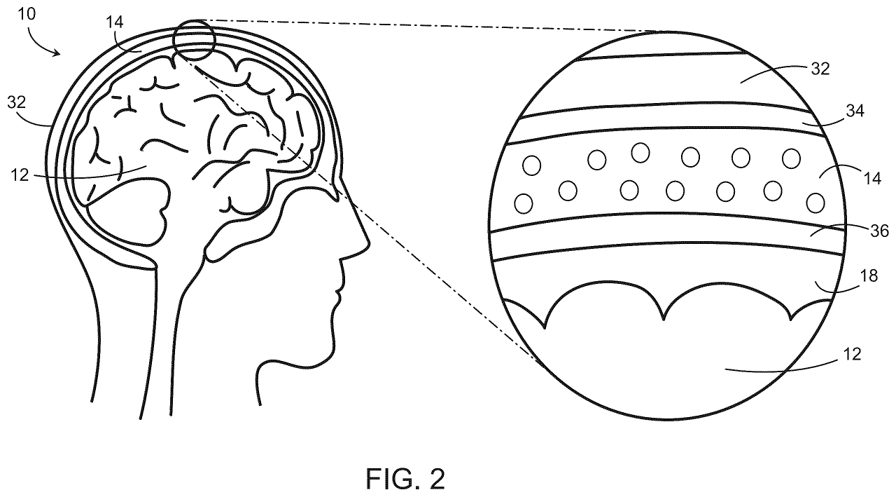

FIG. 2 is intended to show an example of locations in which the creation of a space, pocket, or path can assist in positioning a neural device on or in the tissue of a brain 12 of an individual 10. The magnified section of FIG. 2 represents the various layers of the human head, including a scalp 32, which overlies the periosteum 34, lining the cranium/skull 14. The dura mater 36 is interior to the skull 14 and adjacent to the subarachnoid space 18, which is between the arachnoid matter and pia matter that is exterior to the brain 12. The present disclosure includes vascular access to a region interior to the skull 14, either adjacent to the dura mater 36 or within the subarachnoid space 18 to position a series of electrodes against brain tissue or within brain tissue. In certain variations, the process involves creating a space adjacent to the dura mater 36 to allow an electrode carrier to deploy within the space. Alternatively, as discussed below, deployment of the electrode body can expand, thereby forming the space as the electrodes are positioned.

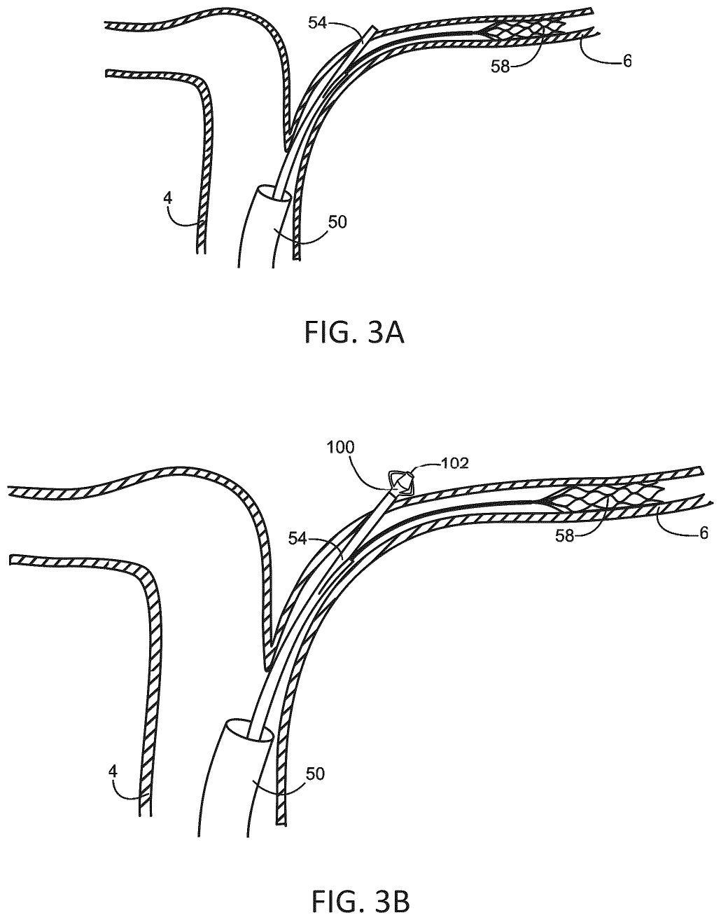

FIG. 3A illustrates an access device 50 advanced into the jugular vein 4 to permit navigation of a catheter 54 within a vessel 6. In this case, the vessel comprises the IPS. As shown, the catheter 54 can include an anchor or stent 58 to secure the catheter 54 at the desired location in the vessel. The catheter 54 can include any number of mechanisms to puncture the wall of the IPS 6 as well as any tissue (e.g., bone, dura, blood vessel, skull, etc.) to provide access to brain tissue. It is noted that any venous and/or arterial approach is within the scope of this disclosure. Moreover, the procedure can include any of the patents discussed above that use the venous network to access brain tissue in order to form a shut to relieve cranial pressure.

FIG. 3B shows a directing structure 100 advanced from the device 50. As discussed below, the directing structure 100 can function to anchor the catheter 54 as well as articulate/rotate to provide navigational capabilities to direct an electrode from an opening 102 in the directing structure 100 to a region of interest within brain tissue. The navigational capabilities can include three-dimensional navigation such as articulation and/or rotation. The directing structure 100 can include any number of steering mechanisms commonly used for articulation of devices, including magnetic positioners, motors, shape memory alloys, pull-wires, a cylindrical cam, etc.

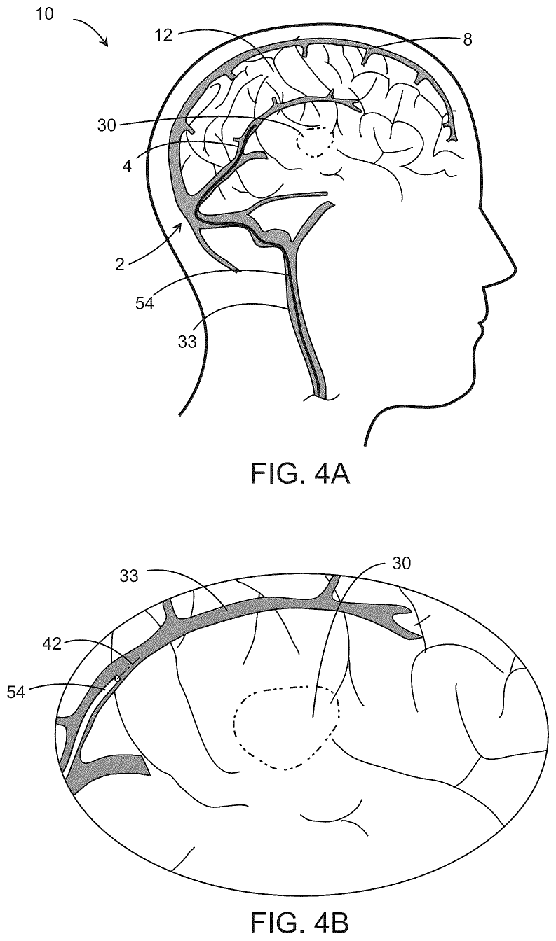

FIG. 4A illustrates a catheter 54 advancing into vessels within the brain 12 of an individual 10 through the jugular vein 4. The illustration shows the catheter 54 advancing into an inferior sagittal sinus 33 towards a target region 30. The catheter 54 can comprise an access catheter or treatment device. In addition, the advancement of the catheter 54, as shown, is for illustrative purposes only. Alternatively, or in combination, a catheter 54 can advance into the superior sagittal sinus 8 or to any other vascular region to navigate towards any target region. FIG. 4A is provided simply as a reference to show a location for subsequently penetrating the vessel, creating a space within the dura, and deploying one or more electrodes or other sensing elements to stimulate and/or sense neural tissue/neural activity. The catheter can be advanced through any vein.

FIG. 4B shows a magnified region of FIG. 4A to better illustrate the catheter 54 advancing in general alignment with an axis 42 of the catheter where the walls of the vessel run approximately parallel to the axis 42. Therefore, any penetration of the vessel wall requires lateral movement of the catheter 54 or other devices used to penetrate the wall.

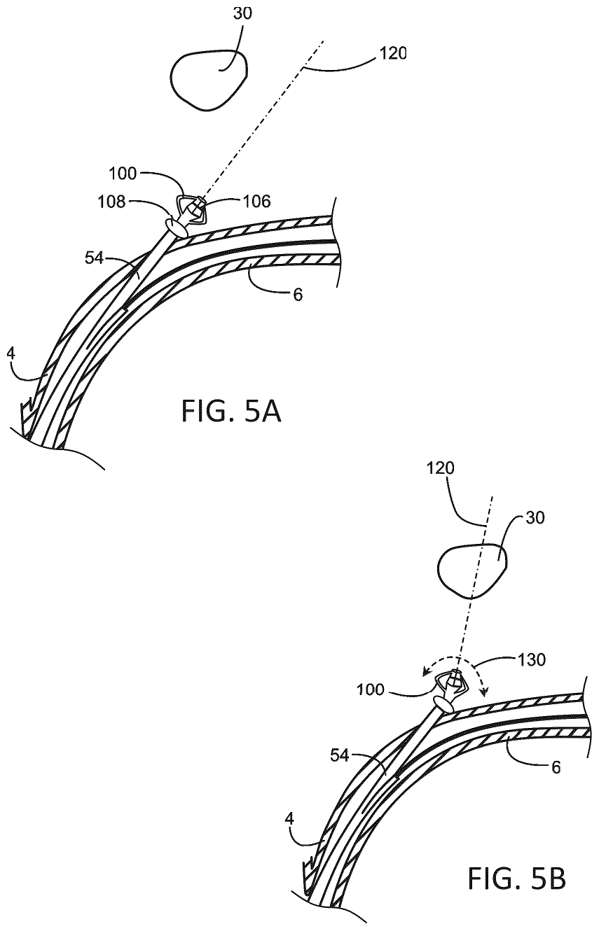

FIG. 5A illustrates a variation of a navigation device 104 similar to that shown in FIG. 3B. The navigation device 104 of FIG. 5A optionally includes a second anchor 108 to allow spacing and/or 3-dimensional movement of the navigation device 104. FIG. 5A also illustrates an exemplary target region 30, similar to that shown in FIGS. 3A and 3B. In operation, navigation device 104 (or another part of the components) will include one or more sensors, beacons, or radiopaque markers 106 that allow a user to identify a potential travel path 120 of an electrode or similar structure that would exit the navigation device 104 in the present orientation. As noted above, the navigation device 104 will include steering features such that it can navigate in a three-dimensional plane, articulate, rotate, or otherwise be repositioned (as shown by arrows 130) such that the potential travel path 120 aligns with a desired region 30 as shown in FIG. 5B. For example, the navigation device 104 can be steered via one or more steering wires. Alternatively, or in combination, the navigation device 104 can be steered using a magnetic field, as discussed below. In some variations of the system, a two-way communication system ensures the navigation device 104 moves in three-dimensional space to a desired orientation. Such a system can include external navigation imaging (e.g., CT scan data, fluoroscopic imaging, ultrasound imaging, stereotactic surgical navigation systems, etc.)

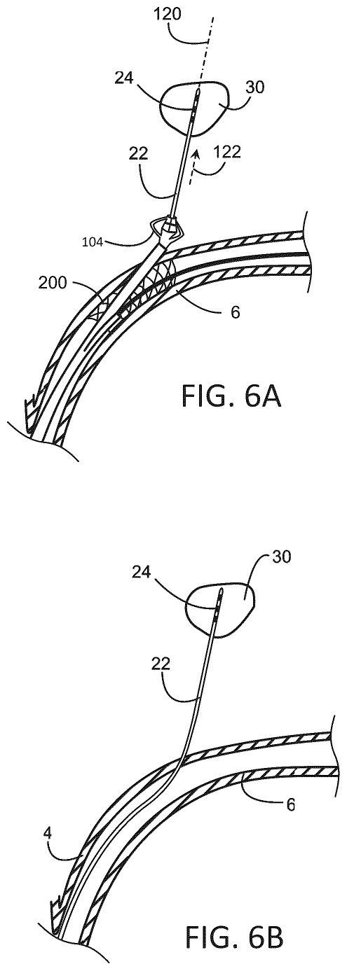

FIG. 6A shows the navigation device 104 in position as an electrode carrier 22 advances in direction 122 along alignment path 120 until electrodes 24 are positioned within or near a region of interest 30. While not shown, the device 104 can advance a needle or other cannula for positioning the electrodes 24 within the region of interest 30. FIG. 6A also illustrates a variation where a stent or stent structure 200 is used within the vessel to assist in transvascular access. Examples of such stents are discussed below.

FIG. 6B illustrates the state where the catheter, navigation device, anchor, and other delivery elements are removed from the site to leave the electrodes 24 positioned within the region of interest 30 and the electrode carrier 22 or lead extending back into the IPS 6 wall and back through the jugular vein 4.

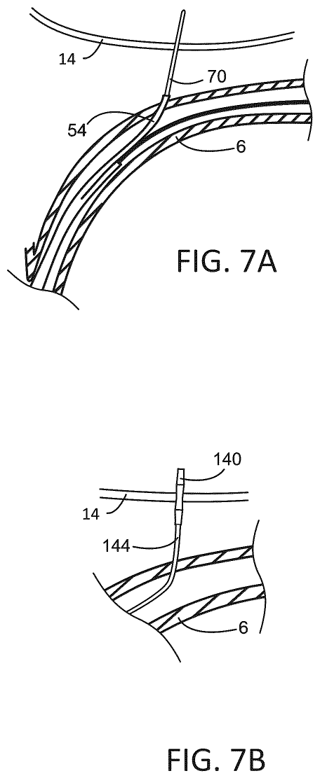

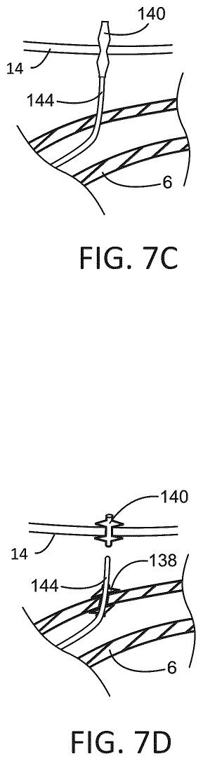

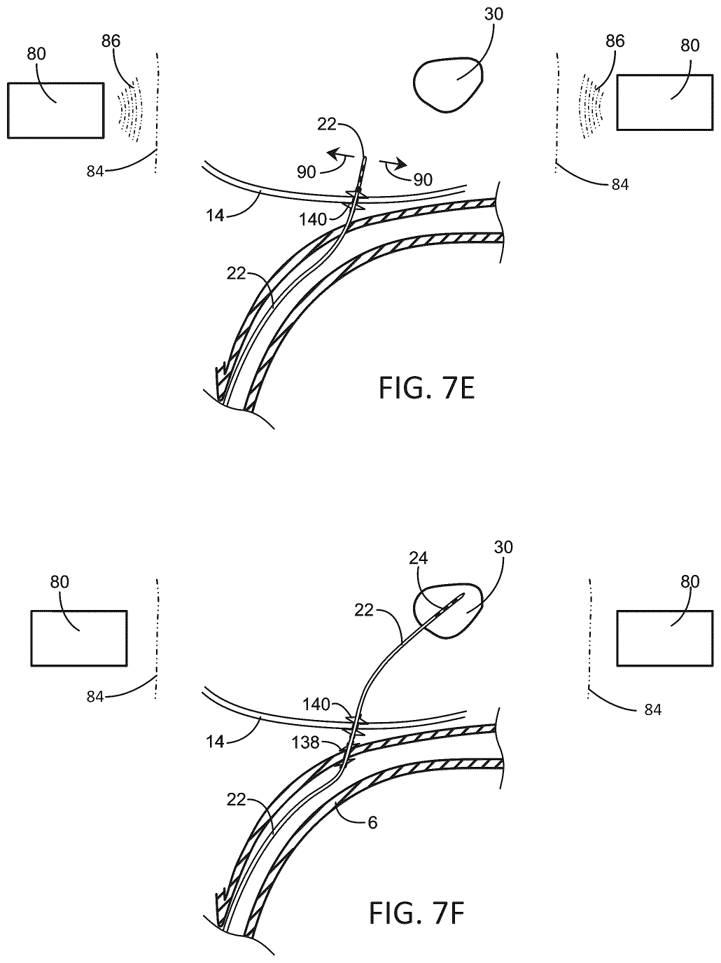

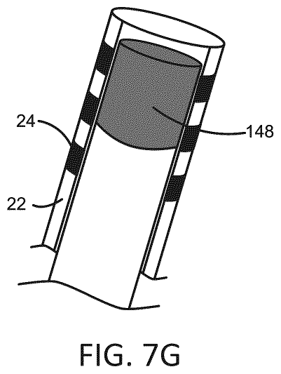

FIG. 7A shows a catheter 54 or similar device within the IPS 6. As shown, one or more anchors can be used to secure the catheter 54 at the desired location in the vessel. The catheter 54 can include any number of mechanisms to puncture the wall of the IPS 6 as well as any tissue (e.g., the skull, etc.) to provide an access to brain tissue. As shown a needle 70 can be used to penetrate the IPS 6 as well as the skull 14. Again, the approach can be a venous and/or arterial approach. In alternative variations, the needle 70 can be used to puncture a bone, dura, or another blood vessel wall.

In some variations, a transdural window mode of delivery can be used in conjunction with a transvenous puncture. A transdural window mode of delivery can comprise accessing the brain through the dura mater.

In some variations, the device can be delivered transvascularly to place electrodes on the surface of the brain in subdural space for the purpose of a sensorimotor neuroprosthesis. In a slightly different variation, the device can be delivered transvascularly to place electrodes on the surface of the brain within a sulcus of the brain, which can be rich brain regions for recording.

FIG. 7B illustrates a deployment stylet 144 positioning a fixation device 140 within the skull 14. In alternate variations, the same catheter 54 can be used to position the fixation device 140.

FIG. 7C illustrates actuation of the fixation device 140 such that a portion of the fixation device 140 deploys about the skull 14. The fixation device 140 can be self-expanding or expand through actuation (e.g., mechanical, electrical, temperature, etc.). Ultimately, as shown in FIG. 7D, the fixation device 140 is in a deployed configuration about the skull 14 to provide a pathway therethrough. It is also noted that in additional variations, a fixation device 140 can be positioned about the vessel 6 wall.

FIG. 7E illustrates the fixation device 140 with an electrode carrier 22 extending therethrough. In this variation, the electrode carrier 22 can be navigated using one or more magnets 80. In one example, the magnets 80 are positioned outside the body, as denoted by lines 84), such that one or more magnetic fields 86 can alter a trajectory of the electrode carrier, as denoted by arrows 90 as the carrier advances distally towards a region of interest 30. Ultimately, as shown in FIG. 7F, the electrodes 24 on the electrode carrier 22 are positioned within or near to the region of interest. FIG. 7F also shows a variation in which an anchor 138 is positioned within the vessel 6 such that there is an anchor 138 and a fixation device 140 both within and outside of the vessel 6.

In some variations, the anchor can be bolstered with one or more of the following: barbs, hooks, tethers, sutures, adhesives sealants, radial force devices, anchoring sutures, expandable anchors, helical coils, spiral designs.

FIG. 7G shows a partial cross-sectional view of an electrode carrier 22 having a magnetic stylet 148 extending within the carrier 22. The magnetic stylet 148 can be removable from the carrier 22 once the electrodes 24 are positioned. The magnetic stylet 148 permits the carrier 22 to remain implanted within the body without a magnetic component. The catheters and navigation devices described herein can also have similar configurations to permit navigation or movement through the use of a magnetic field.

FIG. 8 illustrates the implanted electrodes 24 positioned within a region of interest 30 in the brain and where a lead or electrode carrier 24 extends partially through the vasculature 2 towards the controller 26 such that the stimulation/monitoring device 20 is implanted within the individual while minimizing collateral damage from conventional DBS procedures.

Neurovascular electrophysiology and therapeutic devices are limited in their positioning over or within the cortex by the highly variable physical presence and pathway that veins take. To gain access to wider regions of functionally rich brain regions for recording and/or stimulation purposes, the ability to deploy recording and stimulation arrays without the spatial limitations of the vascular network will prove highly valuable. The ability to safely deliver devices to the same brain regions without the need for a craniotomy is therefore advantageous for patient safety.

The present disclosure also describes methods for the delivery of an electrophysiology (EP) device via the vascular system to the subarachnoid cavity adjacent to cortical areas of interest via transvascular approaches. First, there is a need to access the subarachnoid cavity. There is also a need for a recording array for placement within the subarachnoid cavity. In additional variations, the array can be positioned within other deep brain regions as well.

In some variations, the device can record brain signals generated from the intent to move limbs, including hands, fingers, forearm, elbow, shoulder, head, neck, hips, knees, toes, as well as brow, eyes, tongue, jaw, neck, pharynx, lips or vocal cords. The recording head can deployed in the SSS, which is adjacent to the lower limbs control region of the motor cortex. When the recording head is implanted on either side of the SSS, starting from close to the SSS and then venturing down the lateral veins, the recording head can record from other regions of the homunculus.

In some variations, a method of using electrodes for electrocorticography can be provided and can comprise delivery of electrodes transvascularly and recording either local field potentials or single cell potentials within the extravascular space adjacent to the motor cortex to record motor intent.

The following methodologies are examples of accessing the subarachnoid cavity: access vasculature through standard neurointerventional technique via neck, femoral, or radial puncture; navigate to target puncture site using available neurointerventional imaging modalities, such as C-Arm fluoroscopy; anchor guide within vasculature at target puncture site; puncture through the vessel wall into the subdural space; feed a deployment catheter or device through puncture to subdural space and navigate to deployment site; deploy device, and remove relevant delivery tools.

Although the methods below focus on maintaining vascular blood flow, it is possible to navigate through a sacrificial vessel such as the middle meningeal artery, block the vessel, and follow similar steps of access to the subdural space as an alternative.

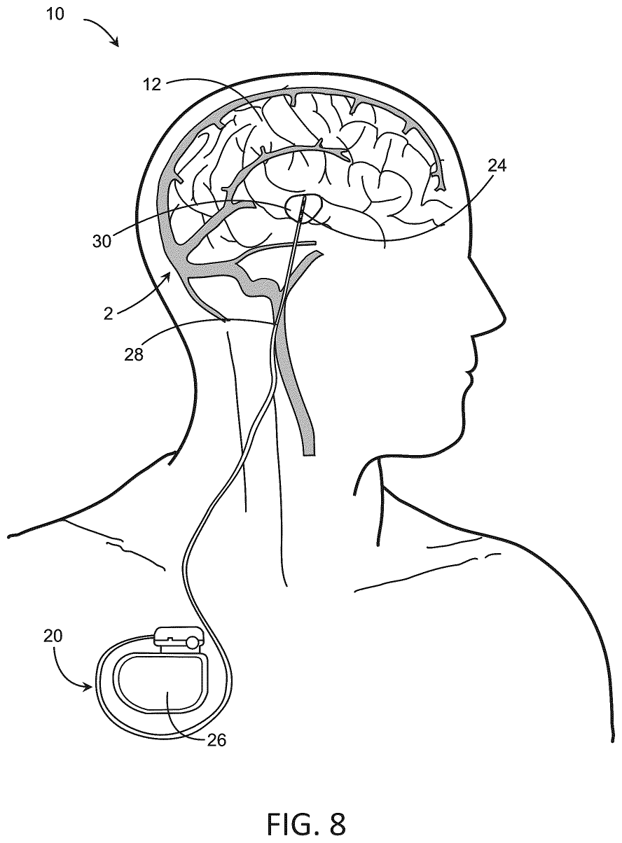

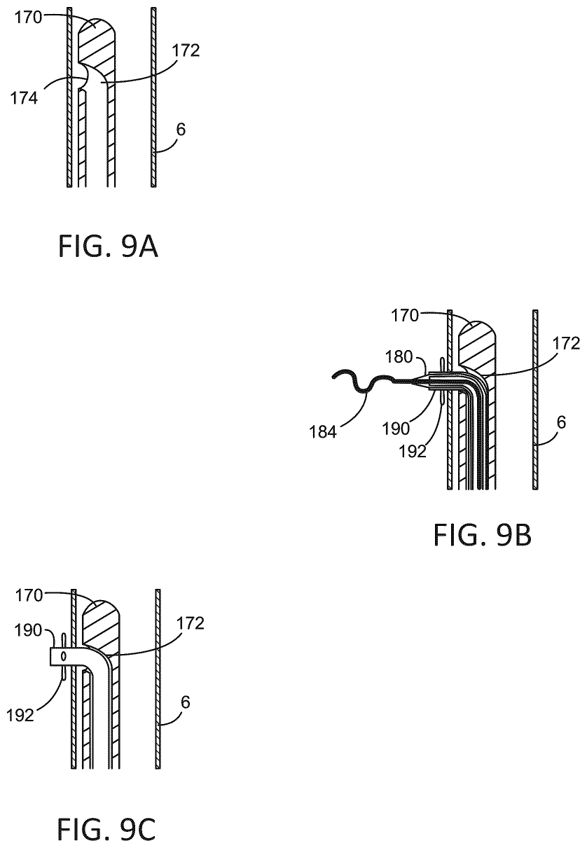

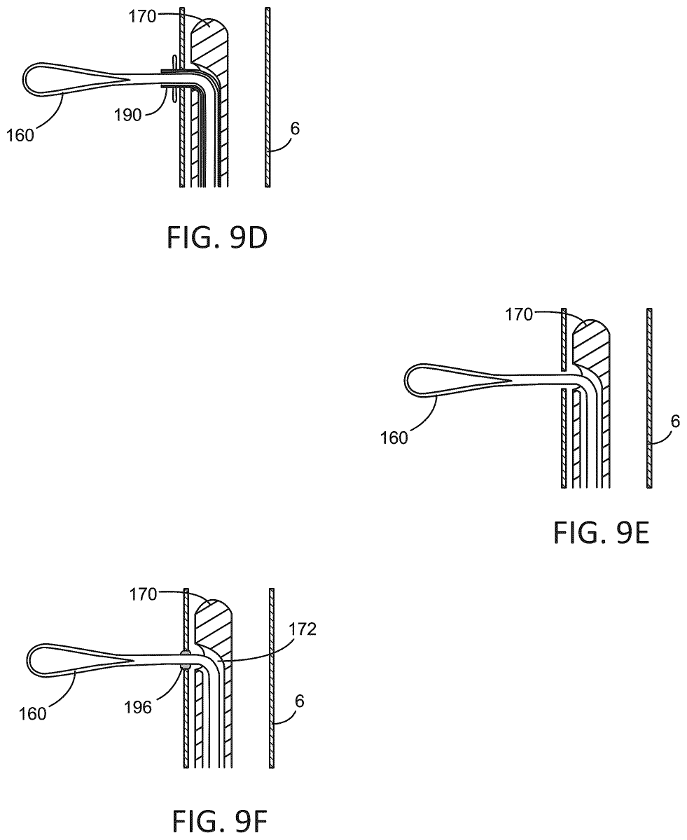

FIGS. 9A to 9F illustrate examples of a process of accessing the subarachnoid cavity or other brain region for delivery of an electrode array through a vessel 6. FIG. 9A shows a directional catheter 170 having a directional lumen 172 that terminates in an opening 174 in a sidewall of the catheter 170. The directional lumen 172 can have any number of deflecting surfaces located therein. Though not shown, variations of a directional catheter 170 can include one more expanding features to secure the catheter within the blood vessel, where such features are disclosed herein, including but not limited to expanding struts/balloon/stent frame, etc.

FIG. 9B shows the directional catheter 170 having an optional guidewire 184 extending through a puncture catheter 180 that extends through the directional lumen 172 and through the opening 174 in the sidewall to puncture a wall of the vessel 6. The puncture catheter 180 can comprise any flexible structure e.g., laser cut stainless-steel (SS), shape memory alloy (SMA), polymeric, etc. In one variation, the puncture catheter 180 comprises a micro hypo-tube catheter, which can be a stainless steel hypo-tube or a hypo-tube comprised of other suitable materials, having a tapered, sharp distal tip for puncturing vascular tissue. The catheter 180 can also include a flexible section achieved via laser cut flexibility features and an internal lumen through which the guidewire 184 can be delivered. As noted above, the guidewires and other devices described herein can incorporate actuatable mechatronic structures that are designed to enhance navigation during minimally invasive endovascular procedures. For example, the guidewires can use shape memory alloys or other actuators to achieve precise bending of at a desired location, including but not limited to the distal end. This bending can facilitate maneuverability through complex vascular pathways. Incorporating SMA allows for a change in shape when an electrical current is applied, causing the SMA to heat and transform into a predetermined shape. Upon cooling, the SMA will return to its original form. Such devices can include a specialized handle that allows practitioners to manage the electrical current delivered to the SMA actuators. By pressing a button, a current is transmitted along the guidewire to its distal end, inducing the desired bend. In addition, the SMA permits bidirectional bending capability for devices.

In the example shown, the puncture catheter 180 is advanced through an intermediate catheter 190. Once the puncture catheter 180 accesses the extra vascular space, a portion of the intermediate catheter 190 is positioned exterior to the vessel 6 wall, and one or more expanding structures 192 are used to secure the intermediate catheter 190 in place. FIG. 9B shows the expanding structures 192 as ribs or a malecot. However, any anchoring structure (e.g., balloons, tines, etc.) can be used.

In some variations, a needle wire is used in place of a guidewire. This allows the operator to advance the puncture catheter 180 into the vessel wall, carefully penetrating through and into the extravascular space using the needle in a similar manner as used in cardiac procedures.

FIG. 9C shows the intermediate catheter 190 after removal of the puncture catheter and guidewire such that the intermediate catheter 190 provides an access path to the extra vascular space. The intermediate catheter 190 can include an outer polymer jacket that deploys the structure 192 by sliding on the intermediate catheter distally, causing the structure 192 to prolapse outward and expand. This expansion can ensure catheter stability for the remainder of the procedure and mitigate the risk that the catheter is accidentally retracted across the puncture point. The fixation fixture may have a flexible polymer skin to form a temporary seal around the puncture site if leaking is present due to a pressure differential between CSF and venous system.

FIG. 9D shows advancement of an electrode carrier 160 into extravascular space through the intermediate catheter 190.

Once the electrode carrier 160 is deployed in the transvascular space through an opening in the vessel, the intermediate catheter can be withdrawn by disengaging the fixation feature for removal from the directional catheter 170, as shown in FIG. 9E.

FIG. 9F shows a variation where the operator can inject a polymer material 196 such as an adhesive (e.g., a cyanoacrylate) through the directional catheter lumen 172, directly onto the puncture site to enhance puncture closure and to secure the position of the transvascular device. After a sufficient time, the directional catheter 170 is removed from the site, leaving the electrode carrier 160 securely deployed in the extravascular space.

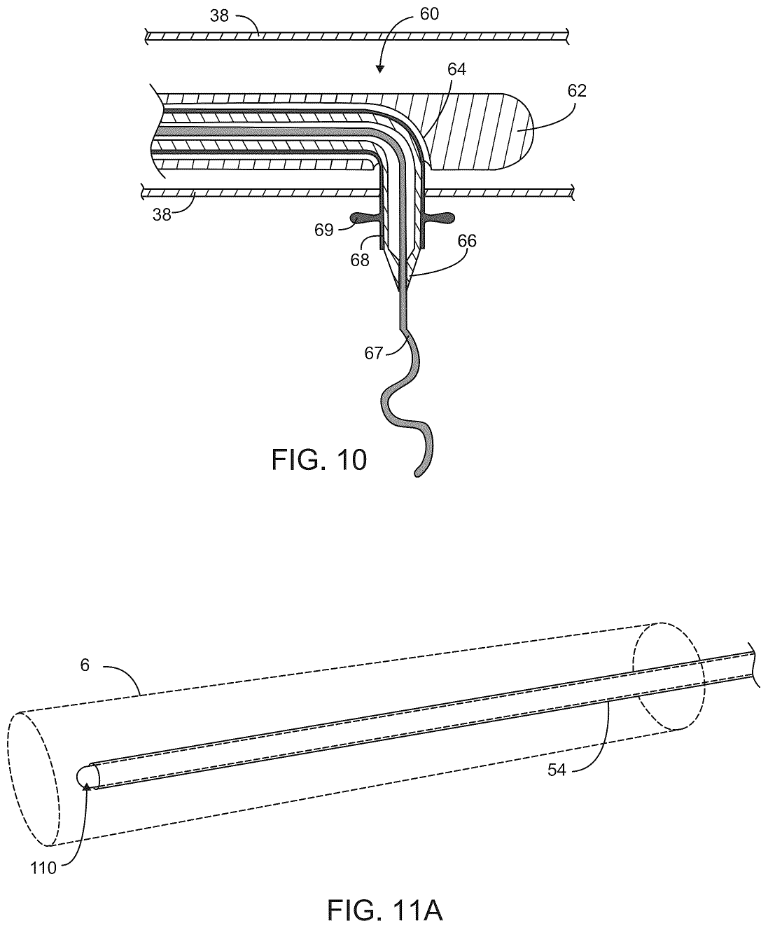

FIG. 10 illustrates one variation of an assembly 60 having a guide catheter 62 with a sideport having a ramp 64 to track a microcatheter 66 and intermediate catheter 68 through a wall of the vessel 6. The intermediate catheter 68 can also incorporate a malecot anchoring feature 69. The intermediate catheter 68 can comprise a soft polymer catheter with an expanding feature (e.g., the malecot 69) at the distal end, which allows for securing the catheter at the site of vascular puncture.

The microcatheter 66 positioned within the lumen of the intermediate catheter 68 can include a tapered, sharp distal tip for puncturing vascular tissue. It may also include a flexible section achieved via laser cut flexibility features to traverse the ramp 64 and an internal lumen through which a guidewire 67 can be advanced or over which the assembly 60 is advanced.

Once positioned, the operator can then deliver devices, catheters, etc., through the vessel wall, as discussed below.

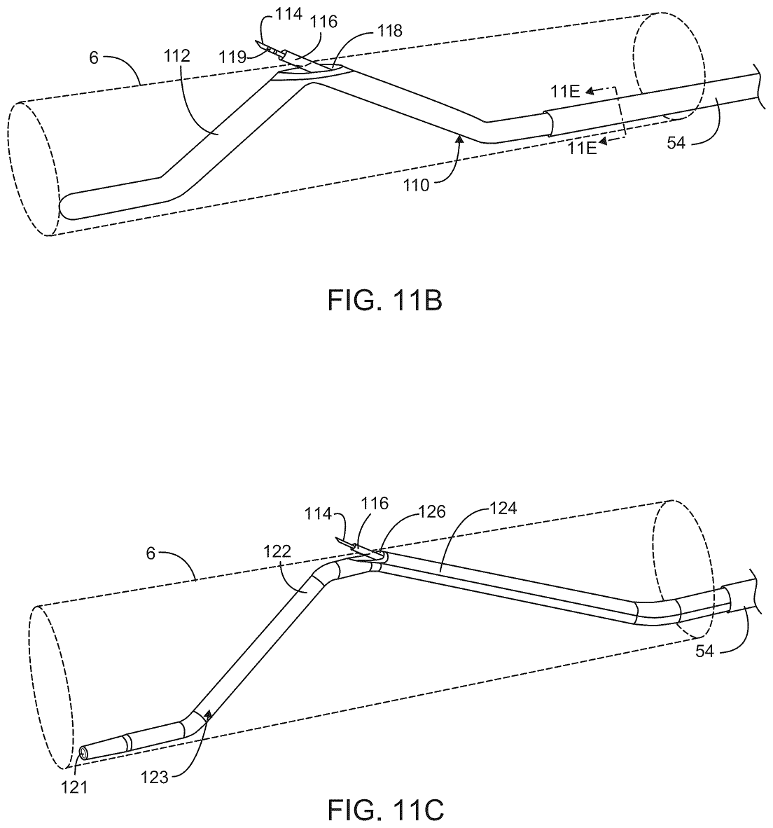

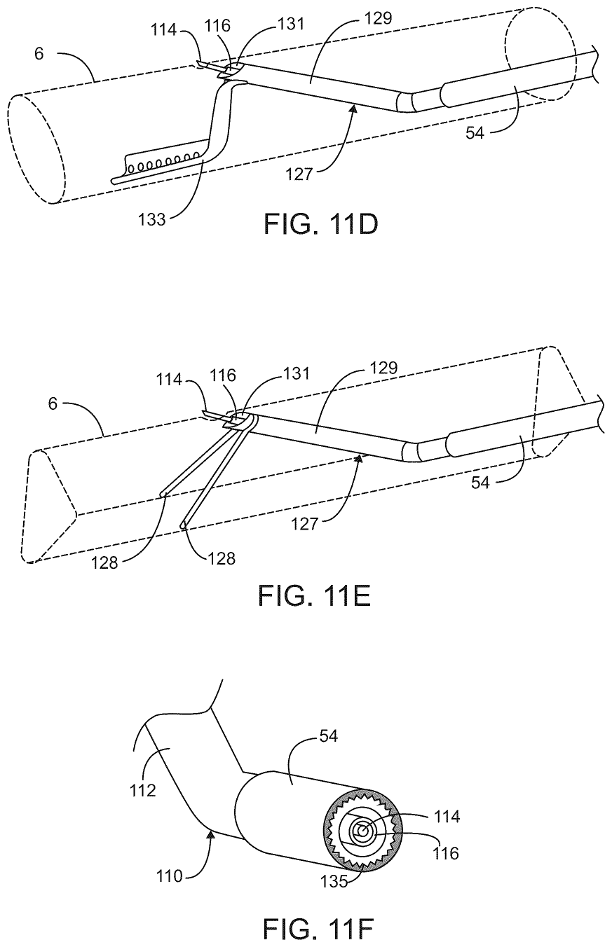

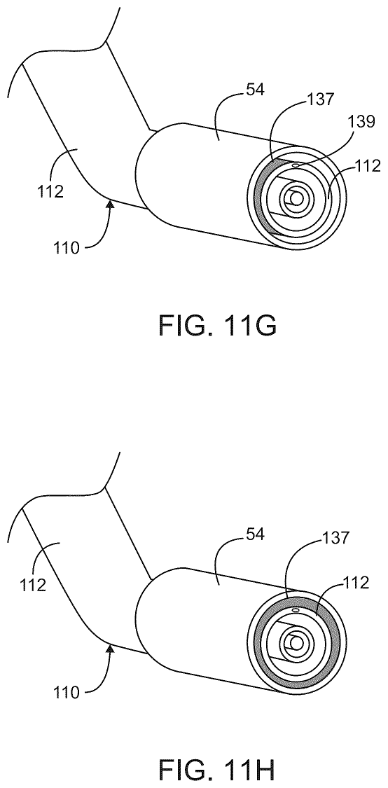

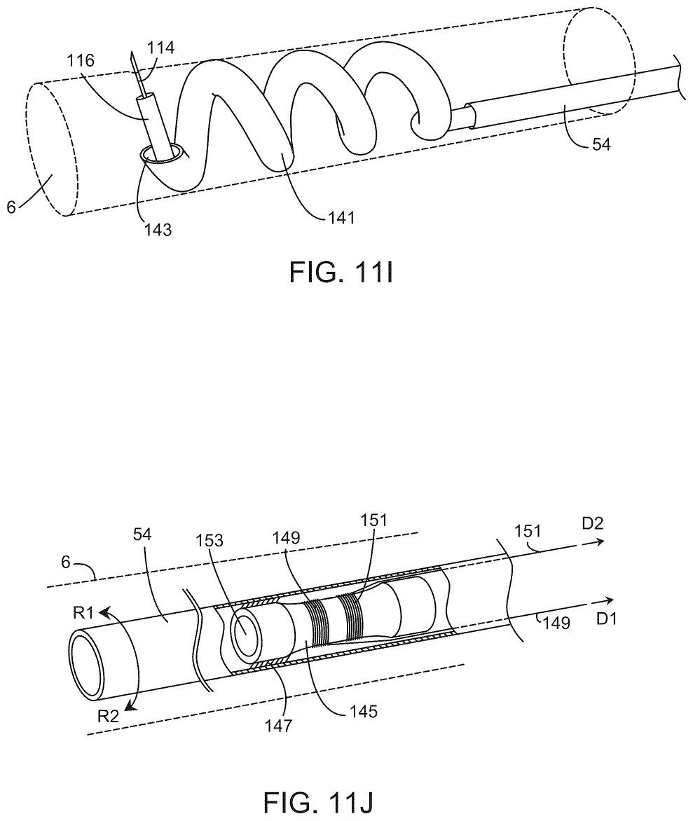

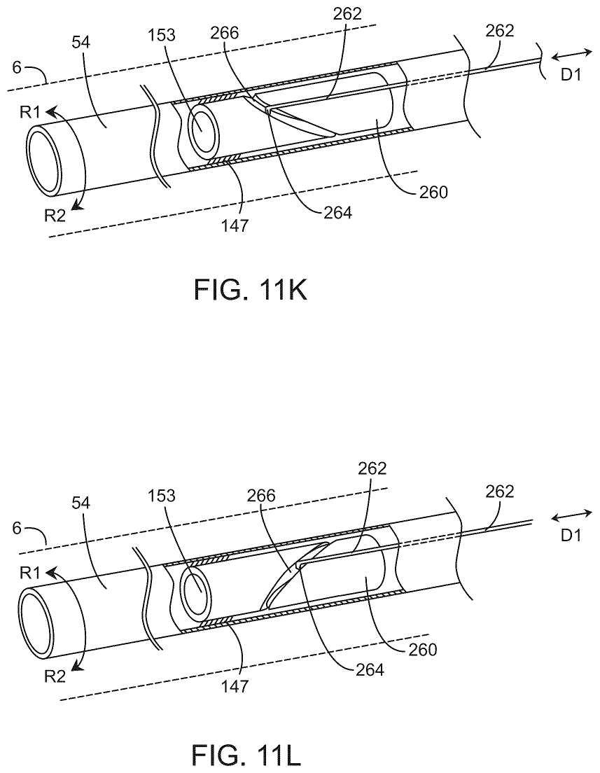

FIG. 11A shows another example of advancing a catheter 54 within a vessel where a secondary piercing assembly 110 is advanced within the vessel in a collapsed state. The piercing assembly 110 can be advanced with the catheter 54 during positioning or can be advanced through the catheter 54 when the catheter 54 is positioned in a suitable location. The piercing assembly 110 can be advanced out of the distal end of the catheter 54, or the catheter 54 can be withdrawn to expose the piercing assembly 110.

FIG. 11B illustrates deployment of the piercing assembly 110 from the guide catheter 54 from the collapsed state to a deployed state. After exiting from the guide catheter 54, the piercing assembly 110 assumes a bend to assume an angled form such that a remainder of the piercing assembly 110 (e.g., the portions distal and/or proximal to the bend) provides apposition against the vessel wall. The piercing assembly 110 can optionally advance over a single guide wire, and it can also be repositioned in-situ. Once positioned, an operator can advance a needle 114 and/or microcatheter 116 to exit the piercing assembly 110 tangentially at an opening 118 on or near the apex of the formed catheter bend. The needle can then puncture through the vessel wall, allowing for a micro-catheter 116 or other devices to advance over the puncture needle 114.

FIG. 11B also shows a variation where a puncture needle 114 includes one or more electrodes 119 that provide impedance or similar electrical information during the puncture procedure. For example, upon penetration of the needle from intra to extravascular space, there can be a measurable change in impedance indicative of penetration of the needle into extravascular space.

FIG. 11C illustrates a variation of a piercing assembly 123 similar to the one shown in FIG. 11B that is positioned outside of the catheter 54. In this variation, the assembly 123 includes a lumen 121 exiting at a distal end as well as a separate tubular structure 124 for advancing a needle 114 and/or microcatheter 116 through an opening 126 in the tubular structure.

FIG. 11D shows another variation of a puncture assembly 127 positioned distally to a catheter 54 within walls of a vessel 6. In this variation, the catheter body 129 of the assembly 127 terminates at an opening 131 that is directed to an apex by a biasing element 133 attached to a distal end of the catheter body 129, which provides a counter force when penetrating the vessel walls. Therefore, the needle 114 and/or microcatheter 116 can advance axially within the catheter body 129. This configuration eliminates the need for tracking a needle or other puncture element through a side port opening on a catheter during post-deployment system withdrawal.

FIG. 11E shows another variation of a puncture assembly 127 positioned distally to a catheter 54 within walls of a vessel 6. In this variation, the vessel 6 comprises a vessel that has a non-circular cross-section, such as the sagittal sinus. Accordingly, the catheter body 129 of the assembly 127 terminates at an opening 131 that is directed to an apex by one or more legs 128 that are straight when located within a catheter but bias outward such that the legs 128 engage into corners or edges of the triangular-shaped vessel. Therefore, the needle 114 and/or microcatheter 116 can advance axially within the catheter body 129. Again, this configuration eliminates the need for tracking a needle or other puncture element through a side port opening on a catheter during post-deployment system withdrawal. It is noted that any number of legs or supports are within the scope of this disclosure.

FIG. 11F is a cross-sectional view taken along the line 11E-11E in FIG. 11B to show a variation of a piercing assembly 110 having an interlocking structure 135 with the interior of the guide catheter 54. This interlocking surface structure 135 can be located on one or more portions of the guide catheter 54 along its length. The figure shows the structure located at the distal end for purposes of illustration. The interlocking feature between the guide catheter 54 and puncturing catheter 112 can comprise any type of interference surface that results in increased rotational stability between the catheters 54, 112. This surface structure 135 allows an operator to “lock in” the rotational alignment of the intermediate catheter, which can improve puncture accuracy by using the mechanical support provided by the guide catheter.

FIG. 11G shows another variation of a piercing assembly 110 having an interlocking structure 137 that locks catheter 112 with the interior of the guide catheter 54. This interlocking structure 137 can comprise an expandable structure such as a balloon and can be located one or more portions of either catheter 54 or 112. FIG. 11G shows the locking structure 137 located on catheter 112 for purposes of illustration. Alternate variations can include an expandable structure on the interior of catheter 54. The locking structure 137 can have one or more lumens 139 that allow for delivery of a fluid into the structure 137 to cause expansion, as shown in FIG. 11H, to provide an interference surface that results in increased rotational stability between the catheters 54, 112. As discussed above, this surface structure 137 allows an operator to “lock in” the rotational alignment of the intermediate catheter 112, which can improve puncture accuracy by using the mechanical support provided by the guide catheter 54.

FIG. 11I shows another example of a catheter 54 positioned within a vessel 6 to deploy a piercing assembly comprising a needle 114 and microcatheter 116. In this variation, the catheter 54 advances a coil-shaped catheter 141 that engages the walls of the vessel 6 to provide stability when puncturing the vessel walls with a needle 114. The coil-shaped catheter 141 is shown to have an opening 143 at a distal end. However, alternate variations allow for one or more openings to be placed anywhere along a length of the coil-shaped catheter 141 such that the microcatheter 116 and needle 114 can exit the opening and puncture the wall while the coil-shaped catheter 141 maintains stability of the devices.

In some variations discussed herein, a catheter or needle device must be rotated within the vessel to penetrate a vessel wall towards a desired direction. In many cases, the working end of the catheter is located vessels within the brain and the proximal portion of the catheter or device that exits the body is rotated. However, attempting to rotate the working end of the device from a proximal end can present difficulties. Accordingly, FIG. 11J illustrates a catheter 54 having a rotational body 145 having a lumen 153 that allows rotation or the application of torque within a vessel 6 either with or without application of torque at a proximal end of the catheter 54. As shown, the rotational body 145 is affixed via fixation 147 to the interior of the catheter 54 and includes at least a first line 149 and a second line 151. The lines 149, 151 are wound in opposing directions about the rotational body 145 such that the application of a force D1 on line 149 rotates the rotational body 145 and catheter 54 in direction R1. Likewise, application of a force D2 on line 151 produces rotational movement of the rotational body 145 and catheter 54 in direction R2. The lines 149, 151 can terminate in a hub or housing at the proximal end of the catheter 54. Alternatively, the lines 149, 151 can terminate along the catheter body and are actuated to produce the pulling forces D1, D2 using any conventional means (e.g., shape memory alloy and electrical current, a separate pull wire, etc.) In some variations, a needle or piercing assembly can be coupled to the rotational body 145 and extend through the catheter. Alternatively, the needle or piercing assembly can be separate from the rotational body.

FIG. 11K illustrates another variation of a catheter 54 having a rotational body 260 having a lumen 153 that allows rotation or the application of torque within a vessel 6 either with or without application of torque at a proximal end of the catheter 54. In this variation, the body 260 comprises a cylindrical cam structure that is affixed via fixation 147 to the interior of the catheter 54. The cylindrical cam structure can include one or more irregular profiles/slots 266 such that a line 262 comprises an end 264 that is nested in the slot 266. The line 262 will be affixed relative to the catheter 54 such that it is limited to linear movement. In this configuration, linear movement of the line 262 results in rotation of the body 260 as the end 264 of the line 262 moves relative to the body 260 but within the slot 266. FIG. 11L shows a state of the device of FIG. 11K during linear movement of the line 262 in a straight line along direction D1.

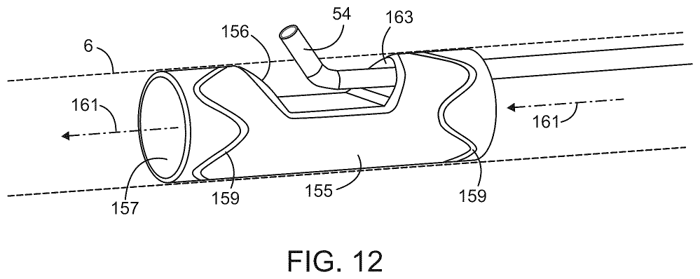

FIG. 12 shows another variation of a diversion device 155 that creates a work region within the vessel 6. As shown, the diversion device 155 diverts blood from the portion of the vessel to be punctured such that blood flow 161 continues through an opening 157 in the diversion device 155 while a ramped surface 156 allows passage of a guide catheter 54 or other structure through the wall of the vessel 6. Such a construction can mitigate the risk of hemorrhagic stroke caused by the injury to the wall of the vessel 6. The device 155 can include a stent-graft type dyneema fabric structure with self-expanding NiTi ribs 159 that conform to the inner diameter of the target vessel 6. The device can be deployed at the target puncture location with the secondary lumen 163 facing the intended puncture location. On deployment, the sinus blood flow 161 is maintained through the larger lumen or opening 157, while a blood-free workspace is provided against the vessel wall. In another variation, the diversion device 155 can comprise a balloon or inflatable structure with one or more channels to achieve diversion of blood flow. In additional variations, flow diverters, such as those shown in FIG. 12 can be combined with the rotational mechanisms described herein to align the secondary lumen and adjacent space in a preferred direction to puncture the vessel at a desired circumferential location within the vessel.

Variations of the methods and devices described herein can be used with echo-navigation to advance the devices through the vessels in the brain. 3-dimensional (3D) transesophageal echocardiography (TEE) is typically used for cardiac catheterization. And uses a matrix-array 3D TEE probe that allows acquisition of high-quality 3D images in real time. Echo-navigation fuses live 3D TEE with fluoroscopy (live X-ray) in real-time. This allows that at any point in time, whether the c-arm moves or the device is navigated to a different part of the anatomy, one can co-register the desired location and co-register the anatomy to create a map, which allows tracing a virtual image based on the patient's real anatomical pathway. In addition, some of the catheters described herein can be configured to adjust a stiffness of the catheter to provide sufficient “pushability” in the catheter in vascular or extravascular space. The variable stiffness catheters can also minimize the catheter steering off track from the target.

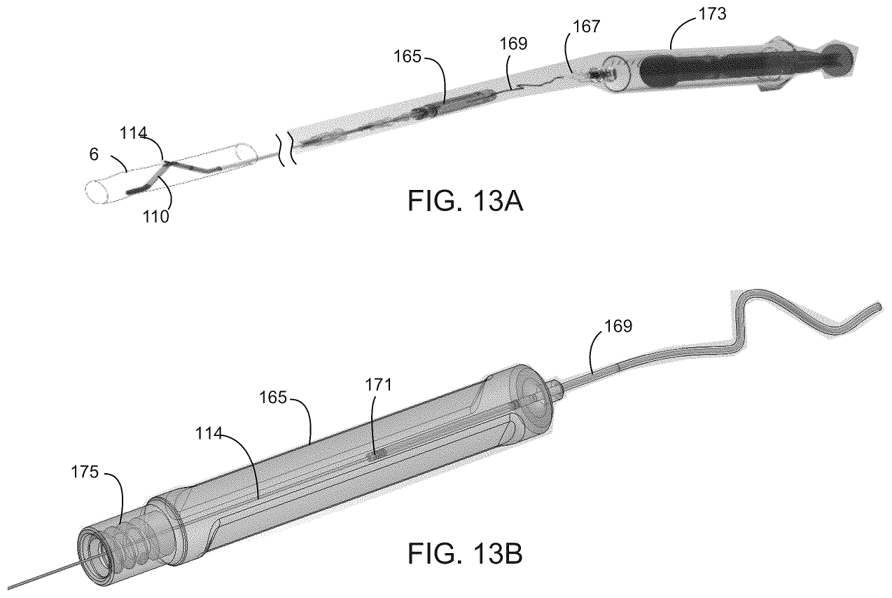

FIGS. 13A and 13B illustrate an example of a controlled system for achieving a precise vessel puncture within the delicate neural anatomy that can be used with any of the devices described herein. The system includes a hydraulic housing 165, shown in FIG. 13B, and fluid connectors such as a threaded luer lock 167, on its proximal end, a high-pressure line 169 having luer lock on the proximal end. The hydraulic housing includes an internal hydraulic piston 171, which is connected to a long puncture needle 114 that extends distally through a series of water-tight seals. To use the device, an operator advances the needle 114 into a piercing assembly 110 and secures the luer lock thread 175 to the proximal end of the catheter. The high-pressure fluid line 169 can be connected to a fluid source such as a balloon indeflator device 173 filled with saline or other fluid. When puncture is required, the operator increases hydraulic pressure in the indeflator device 173, which in turn advances the hydraulic piston 171 to advance the needle 114. The travel distance can be limited through the use of various stops to allow for very tightly controlled parameters. Further, the operator may be able to visualize the precise moment of puncture by monitoring a pressure gauge or indicator on the indeflator device 173, which may show a marked drop in system pressure as the needle penetrates the vessel/dural tissue.

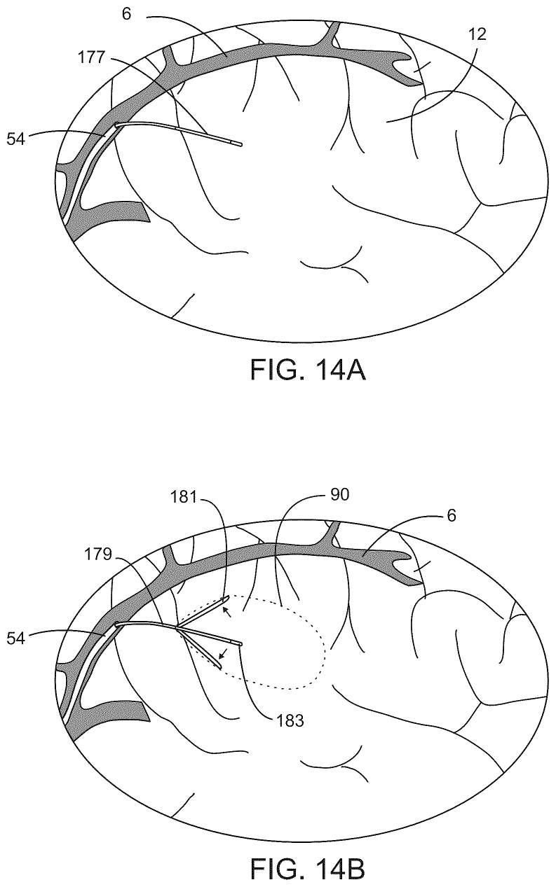

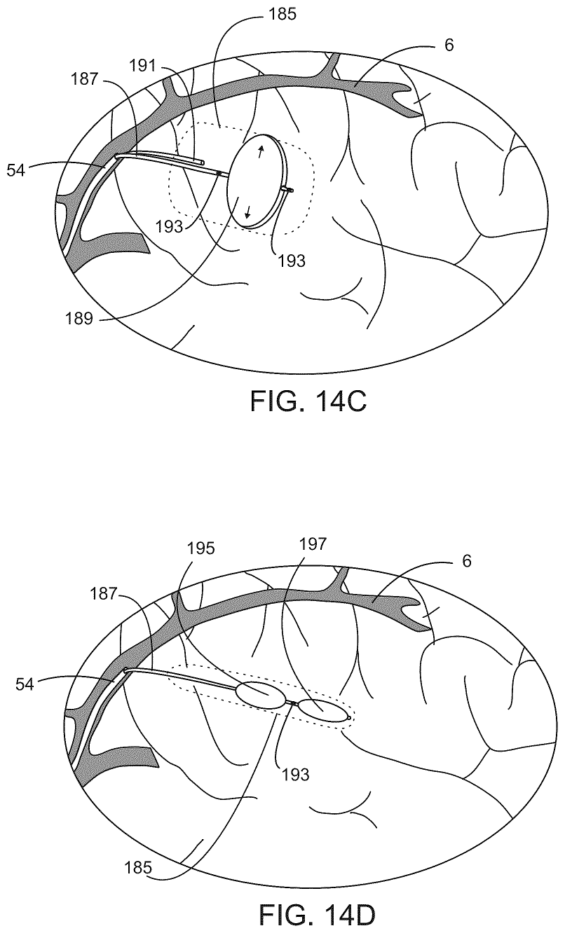

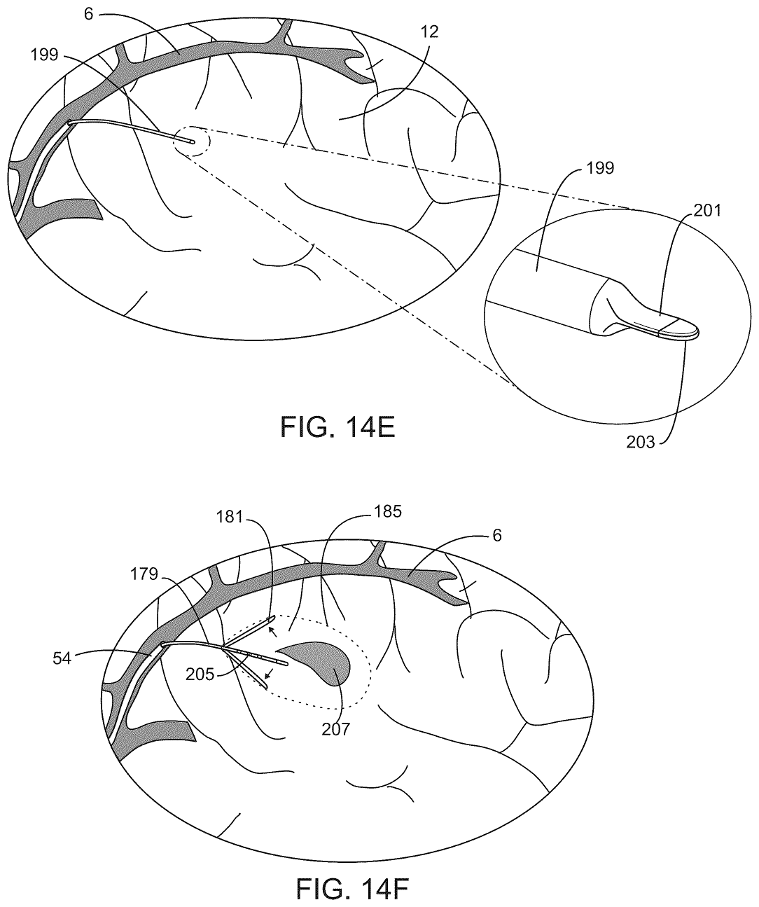

FIG. 14A is an illustration showing a state after a wall of the vessel 6 is punctured as discussed above, and one or more catheters or devices 177 are advanced out of the vessel 6 to prepare for deployment of electrodes on or within brain tissue. In some variations, it is desirable to form a cavity or space between the dura and brain tissue 12, which assists in positioning of the electrodes over or in tissue. In one variation, one of the devices 177 can include a balloon member that expands to create a track between the dura and brain tissue 12, where the track is used to navigate the catheter (or a separate catheter) to a desired target location on or adjacent to brain tissue for deployment of electrodes.

FIG. 14B illustrates one variation of a device 179 having forceps-type blunt jaws 181 at the distal end. These jaws 181 can be actuated by the operator, as shown by the arrows, outwardly moving and gently dissecting dural tissue to create a space, pocket, or cavity 185. The device 179 can be moved over various locations to create a sufficient cavity 185. In most cases, dissection of the tissues, such as the dura or other tissues in the subarachnoid space, occurs tangentially to the brain tissue to minimize compression of the brain tissue. The center portion 183 of the device 179 can be used for impedance measuring to determine whether the tissue is sufficiently separated to create the cavity 185. Alternatively, a separate electrode wire can be advanced from the center portion 183. In some variations, impedance measuring can be performed by the device 179, including jaws 181 and center portion 183.

In an additional variation, the device 177 can include one or more electrodes that provide impedance or similar electrical information during the puncture procedure. For example, upon penetration of the needle, there can be a measurable change in impedance indicative of penetration into extravascular space. The impedance information recorded from catheter can then provide real-time feedback to the physician on what type of tissue the puncture system is exposed to at any time to guide the procedure. As well as real-time visualization, either laser, camera, optical coherence tomography (OCT), and/or ultrasound (US) as the device enters the subdural space.

FIG. 14C shows another variation where the device comprises a balloon catheter 187 with a 2D “pancake” profile balloon 189 that generally expands in a larger lateral direction, as shown by arrows, versus a height-oriented direction. Upon expansion in the sub-dural space, the balloon 189 acts to separate tissue and create a cavity 185 for deployment of an electrical array. As noted, the balloon can create a single cavity or can move about to increase the size of the cavity 185. FIG. 14C also illustrates a variation of the method of creating a cavity 185 and using a visualization device 191 adjacent to the balloon 189. Such a visualization device 191 can be used in any variation discussed herein.

FIG. 14D shows another variation where the device comprises a coaxial or axial balloon catheter 187 having any number of balloons. For purposes of illustration, the catheter 187 shown includes a first balloon 195 and a second balloon 197. These balloons 195, 197 can be coaxially located, e.g., balloon 197 advanced within a lumen or passage of balloon 195. Alternatively, the balloons 195, 197 can be axially spaced on the catheter 187. As illustrated, the balloons can make a narrow pocket by sequential dilation. For example, a smaller balloon 197 can displace the dura or other tissues to create space for a wider/larger balloon 195 to size the pocket or a dimension of the cavity 185 accordingly.

FIGS. 14C and 14D also illustrate another feature of the methods and devices where one or more electrodes 193 can be used to determine whether the cavity 185 is created. In one example, the electrodes 193 can measure electrical impedance of adjacent tissue. When the tissue is separated during creation of the cavity 185, the electrodes will be separated from the tissue and can confirm creation of the cavity 185. In some variations, an electrode can be positioned on opposite sides of the device, e.g., one adjacent to the brain tissue, and one on an opposing side of the device. Alternatively, an electrode or other sensor can be advanced separately to confirm creation of the cavity 185.

FIG. 14E illustrates an optional device 199 that can be advanced adjacent to brain tissue 12 to separate the dura and either create a pocket through blunt dissection or to create a path for the devices described herein. As shown, the device 199 can include a wedge or duckbill portion 201 that separates and dilates tissue in the extravascular space. The wedge portion 201 can include a tip 203 having flexible or expansive (e.g., a balloon) properties relative to the remainder of the wedge portion 201.

FIG. 14F illustrates an additional aspect of a method of creating a space or cavity 185 within or adjacent to dural tissue. In this variation, the device 179 can include one or more ports 205 that deliver a contrast agent 207 to the space. In additional variations, the contrast agent 207 can be delivered by the catheter 54, the balloons or the blunt of the forceps-type blunt jaws devices discussed herein. The contrast agent 207 allows visualization through non-invasive means to determine whether the cavity 185 is created. Ports that deliver contrast agents through the blunt jaws when in an open configuration as a method of confirming the location of blunt jaws with digital subtraction angiography and radiocontrast where the contrast highlights the geometry of the brain surface and makes it visible to the operator.

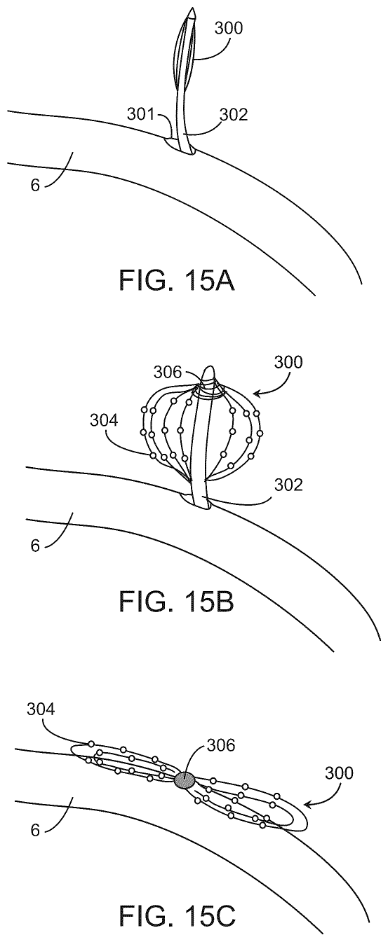

FIGS. 15A to 15C illustrate another variation of an electrode device passing through an opening 301 in a vessel. As shown in FIG. 15A, the device 300 can be positioned on a catheter 302 for advancement out of the vessel. FIG. 15B shows the device 300 in an expanded state such that a number of electrodes 304 spread apart on the device 300. However, the device 300 can also include a sealing member 306 at a distal portion. FIG. 15C illustrates a state where the catheter 302 (FIG. 15B) is withdrawn into the vessel 6 causing the device 300 to flatten about the vessel 6 and where the sealing member 306 seals the opening in the vessel 6 to allow for hemostasis.

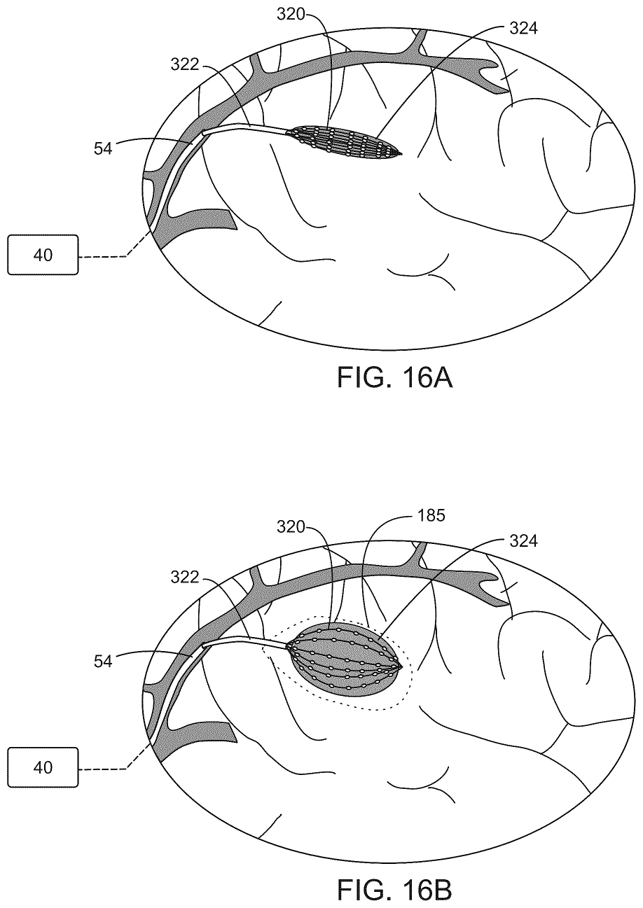

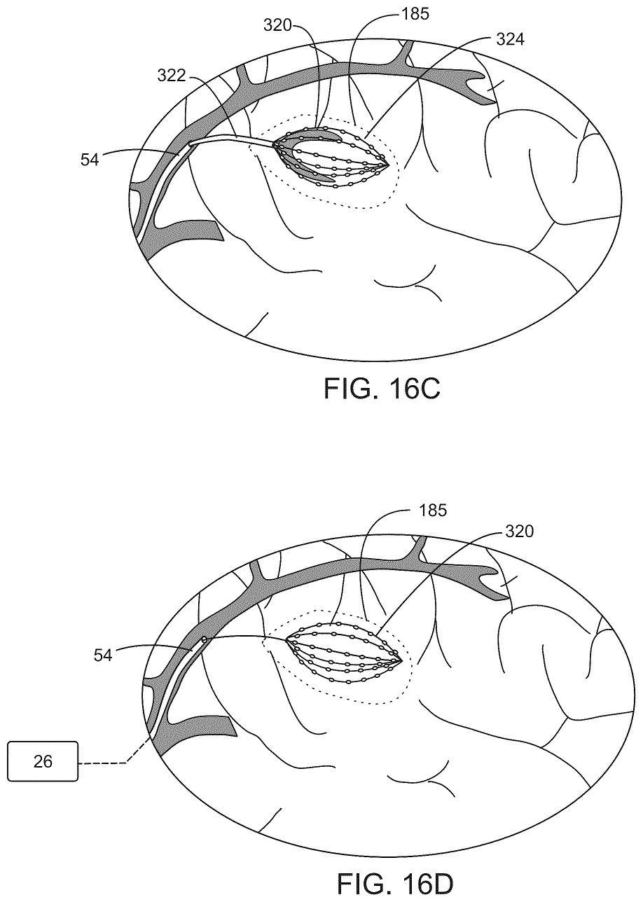

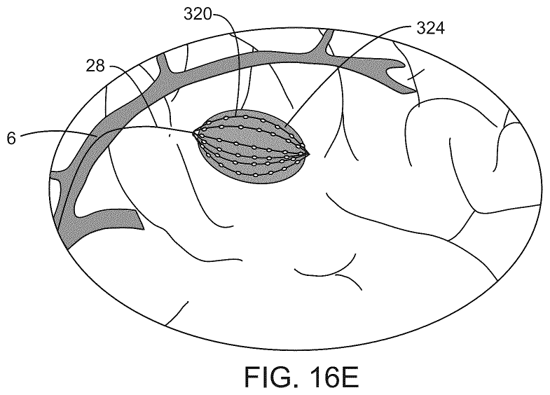

FIGS. 16A to 16D illustrate another variation of an electrode device 320 coupled to a balloon catheter 322. As shown in FIG. 16A, the electrode device 320 can advance outside of the vessel and adjacent to tissue either before or after a cavity is formed. FIG. 16B illustrates expansion of the balloon 324, using an inflation source 40, causing the electrode device 320 to expand, which can also optionally create the cavity 185 as discussed above. The electrode device 320 can be coupled to the balloon catheter 322 or balloon 324 using any remotely detachable construction. Once the electrode device 320 is deployed, as shown in FIG. 16B, the balloon 324 can be withdrawn or inverted within the electrode device 320 and back into the balloon catheter 322 leaving the electrode device 320 within the cavity 185. As shown in FIG. 16D, the electrode device 320 remains deployed within the cavity 185 and the electrode device 320 remains coupled to a controller 26 or other power supply while the inflation source is disengaged.

In another variation of the device and methods described herein, the balloons can serve as scaffolding structures for the electrode assemblies. The balloons can be permanent implants or resorbable. FIG. 16E shows one example, similar to the deployment of the balloon 324 as described above. However, in this variation, the balloon 324 is detachable from the catheter 322 (see FIG. 16A), which may be steered using the catheter to achieve a desired position. Accordingly, the catheters 54, 322 (see FIG. 16A) are removed leaving the electrode assembly 320 located over a balloon 324 (either deflated, partially deflated or fully inflated) where a lead 28 extends from the electrode assembly 320 through and the vessel 6. Variations of the balloons can include balloons coated or filled with agents to promote healing or enhance signal transmission.

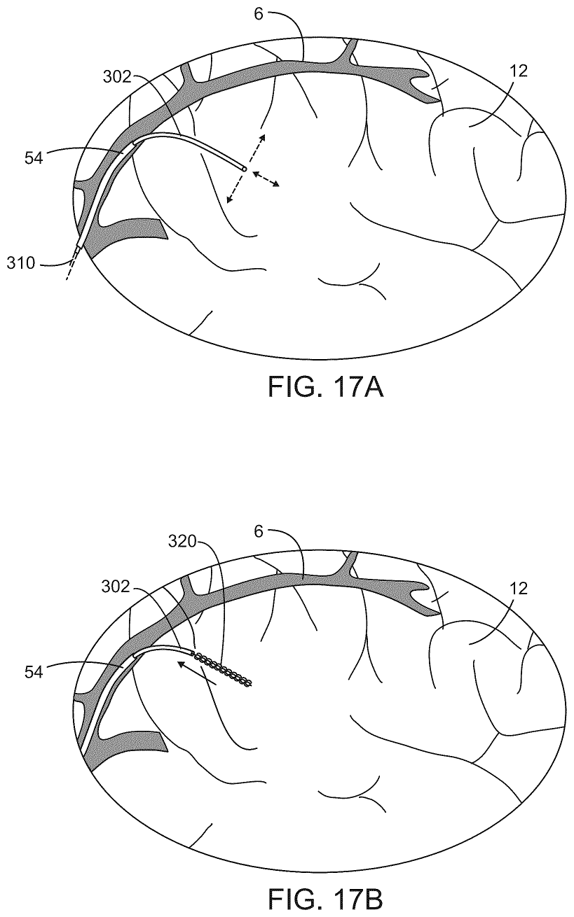

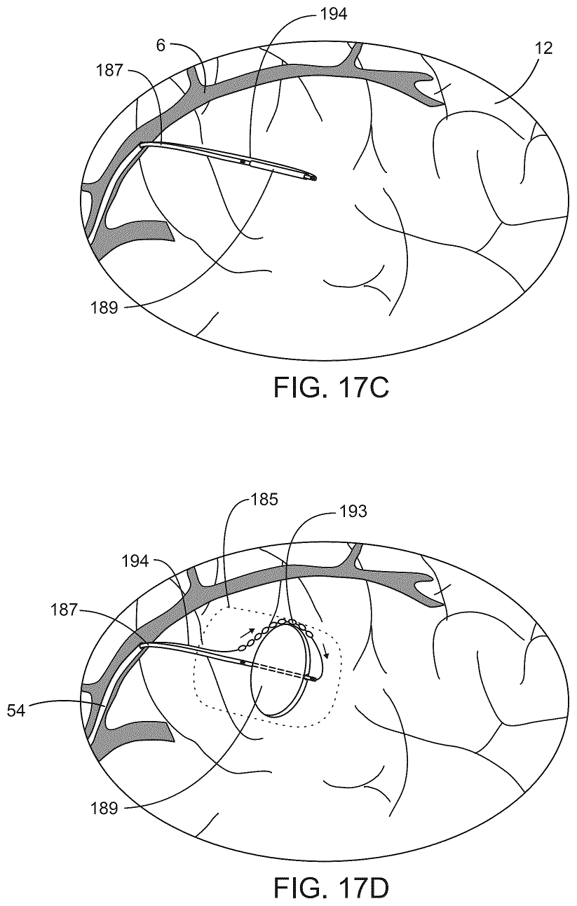

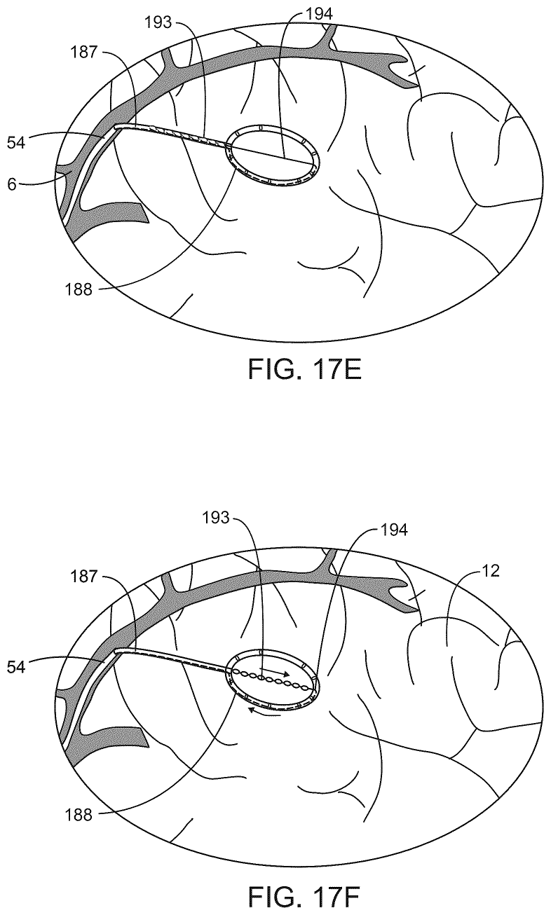

FIG. 17A illustrates an additional concept of placement of electrodes or an electrode array at a desired region of tissue using improved control mechanisms. FIG. 17A is an illustration showing the punctured wall of the vessel 6 punctured as discussed above and one or more catheters or devices 302 are advanced out of the vessel 6 to prepare for deployment of electrodes on or within brain tissue. As noted above, in some variations, it is desirable to form a cavity or space between the dura and brain tissue 12, which assist in positioning of the electrodes over or in tissue. In the illustrated variation, the catheter 302 is steered using any number of pull wires 310 to reposition the catheter tip as shown by the arrows. Alternate steering configurations are also within the scope of this disclosure. The catheter 302 can have radiopaque features or other features to allow a physician to determine that the position of the catheter 302 is in a desirable location. Once the position is confirmed, as shown in FIG. 17B, the catheter 302 can be withdrawn in a manner that leaves the electrodes in the desired location and deployed/expanded if necessary.

FIG. 17C illustrates another variation where a catheter 187 is advanced from the vessel 6 to position a balloon 189 over brain tissue 12. As discussed above, the balloon catheter can be expanded to create a space 185 between the dura and brain tissue 12. In this variation, a wire 194 also extends adjacent to the catheter 187 such that it returns through a passage of the catheter 187. FIG. 17D illustrates expansion of the balloon 189 to create the space 185. The wire can be pulled proximally through the balloon 189 to pull electrodes 193 (or an electrode array) from a catheter 54 into a desired position. In this variation, the balloon 189 can be fully or partially expanded to aid in positioning of the electrodes 193. After the electrodes 193 reach a desired location, the wire 194 can be detached from the electrodes 193 through any number of conventional detachment mechanisms (e.g., mechanical, chemical, electrolytic detachments, etc.)

FIGS. 17E and 17F illustrate another example of pulling electrodes 193 into place using a wire 194. In this variation, the catheter 187 comprises a double-balloon 188 system where the double-balloon 188 expands to first create the space. The electrodes 193 can remain in the shaft of the catheter 187 and are coupled to a wire 194. FIG. 17F shows the wire 194 being pulled to draw the electrodes 193 out of the catheter 187 and into position over brain tissue 12.

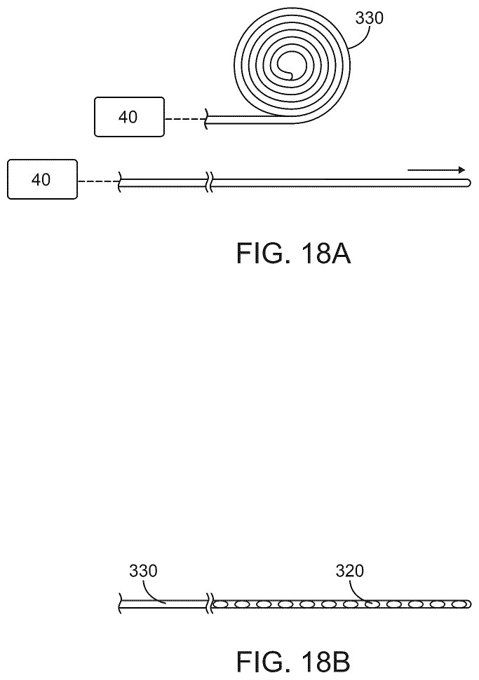

FIGS. 18A and 18B show another variation of a balloon catheter that can be positioned between brain tissue and the dura. In this variation, the pressurization of the balloon 330 from an inflation source causes the balloon 330 to unroll in a distal direction shown by the arrow. Unrolling of the balloon 330 allows positioning of the balloon 330 in a desired location. FIG. 18B shows an electrode array 320 that can be attached to an exterior of the balloon 330 or within the balloon 330 such that the balloon 330 is used to pull the electrode in place as described above. As noted herein, the disclosed balloons can be bioabsorbable or can remain implanted to provide support for the electrode device.

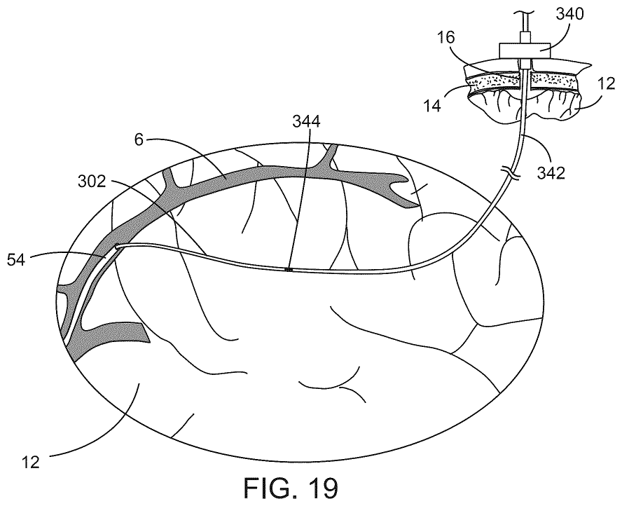

FIG. 19 illustrates another means of positioning a catheter 302 out of the vessel 6 and adjacent or into brain tissue 12 that can be combined with any of the variations described herein. In this variation, a burr hole 16 or other opening is created in a skull 14 of the individual, and a positioning catheter 342 is passed through the hole 16 (optionally, a port 340 can be used to create/access the brain tissue through the skull). The positioning catheter 342 can be steerable or remotely steerable and can include any number of coupling structures 344 to pull the catheter 302 into a desired location. Upon proper placement, the positioning catheter 342 disengages the catheter 302, and the positioning catheter 342 is removed along with the port 340.

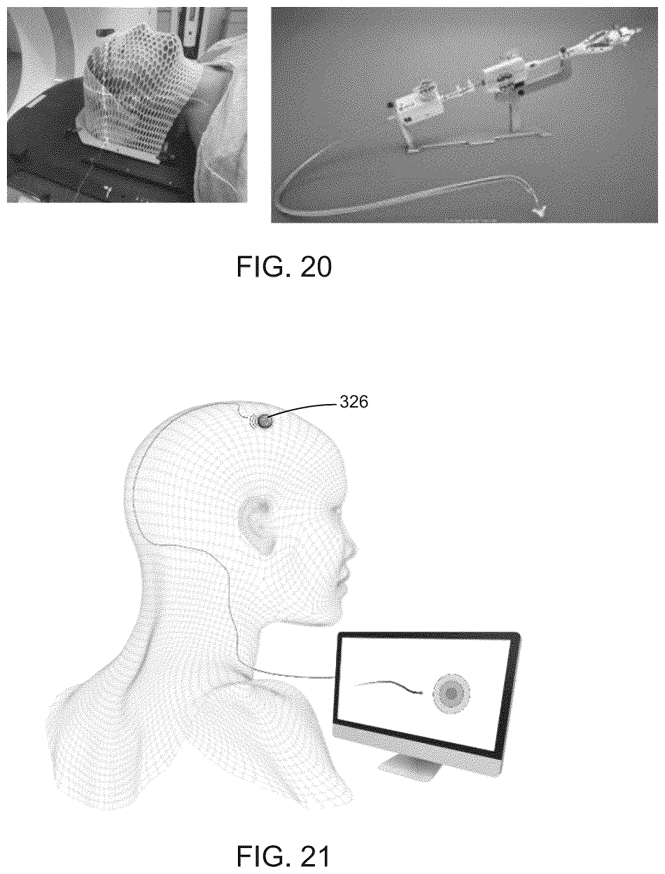

FIGS. 20 and 21 illustrate additional systems for use in delivering the electrode devices to the desired target regions.

FIG. 20 illustrates a stereotactic, intraoperative stereotactic fixturing system that keeps the relative positions of the patient's head and catheter system in known alignments. In one variation, this system requires imaging inputs. The head fixturing may be similar to patient-specific fixtures used in targeted radiotherapy, with the addition of a connection point at which a catheter cradle system can be positioned at a known alignment with the head and neural target. This may allow for higher accuracy in positioning and alignment during the procedure, especially during critical puncture and extravascular tracking steps.

FIG. 21 shows the use of an external electrode 326 or an array of electrodes that are precisely aligned on the scalp at the specific target region of the cortex, achieved through CT/MRI/fMRI methods (similar to radiotherapy targeting). The external electrode 326 is then used to drive a signal intraoperatively, which can be detected via an electrode placed on a guidewire or catheter (not shown). As the wire or catheter is advanced across the brain surface, the signal strength of the external electrode is used to guide the internal system to the correct target with reduced reliance on imaging.

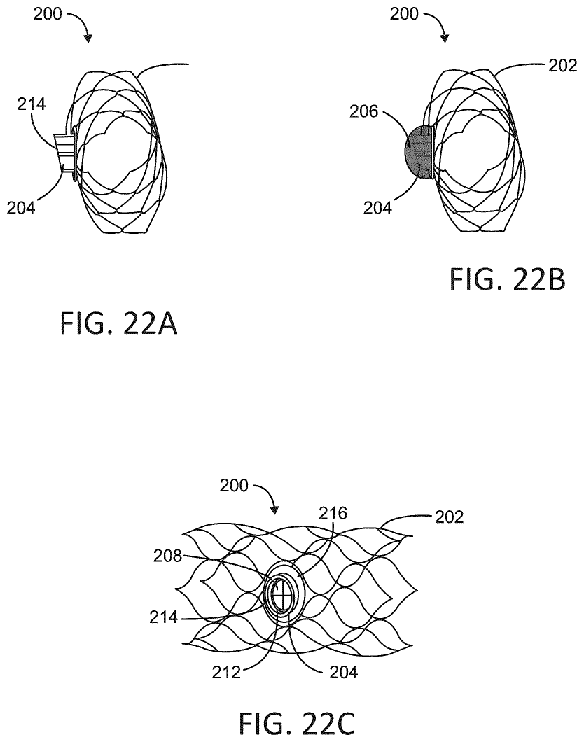

FIGS. 22A to 22C illustrate an example of a stent structure 200 having a stent body 202 that can be positioned within a vessel. The stent body 202 can include a port 204 having a lumen 212. In one variation, an edge 214 of the port 204 that is opposite to the stent body 202 comprises a sharp edge transitioning to a medical textile brim section 216 on the proximal side or stent side. The port 204 includes a lumen 212 and can optionally have a valve or rubber seal component 208 included to mitigate transgression of fluid across the open port lumen. In one variation, as shown in FIG. 22B, the port 204 includes a bio-dissolvable or degradable material that encases the port 204 or over the sharp edge 214. This material can be configured for slow or fast dissolving (e.g., order of ˜2 weeks) within the bloodstream to allow a controlled exposure rate of the sharp leading edge 214 of the port 204 to a wall of the vessel. The stent body 202 can be designed to provide constant outward radial force on the port 204, allowing controlled penetration of the port through the vascular tissue. A guide tube may be connected to the puncture lumen, trailing through the vasculature to the puncture site to aid later navigation and deployment through the puncture. Once the operator gains access to the target vessel with a guide catheter, the operator delivers the port stent device through the guide catheter, carefully positioning the port feature at the target portion of the vein where extra vascular access is required.

The operator deploys the stent port (self-expanding or balloon expanded), then removes the guide catheter and ends the procedure.

Either immediately or after a period (e.g., 2 weeks) CT or similar imaging protocols can be used in a follow-up procedure to deploy a transvascular device through the port and into the extra vascular space. If a guide tube is present, the transvascular device is fed through the tube to the port. If a guide tube is not present, standard neurointerventional techniques would be used to navigate to the port.

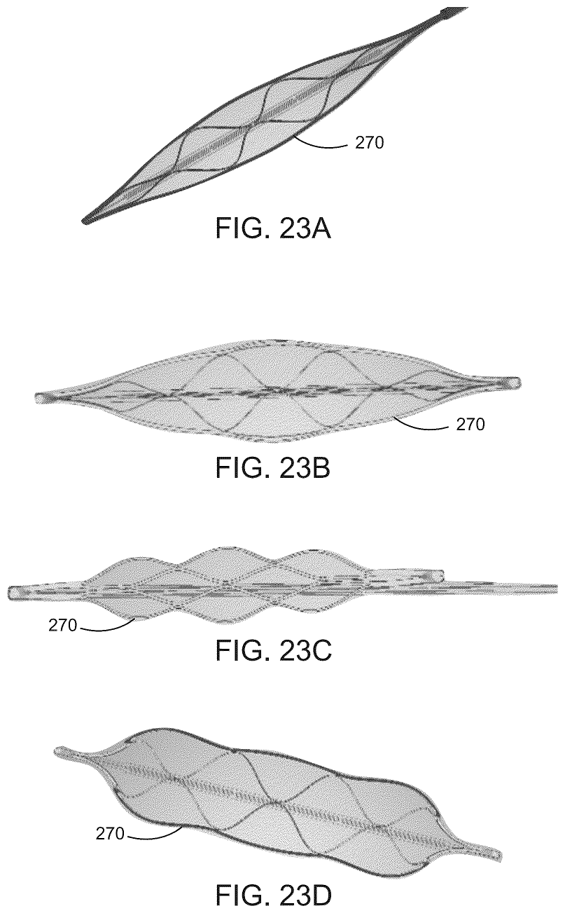

FIGS. 23A to 23D show various examples of stent scaffold style 2D self-expanding arrays 270. These arrays 270 can comprise a superelastic or shape memory alloy and can have electrodes along portions of the stent structure or integrated into the stent structure. However, unlike typical stents, these stent arrays 270 conform to a thin “paddle” arrangement for delivery to a target. After being unconstrained, e.g., exit from a restraining catheter, the stents expand primarily in a direction that is lateral to brain tissue to assume the 2D geometry as discussed above.

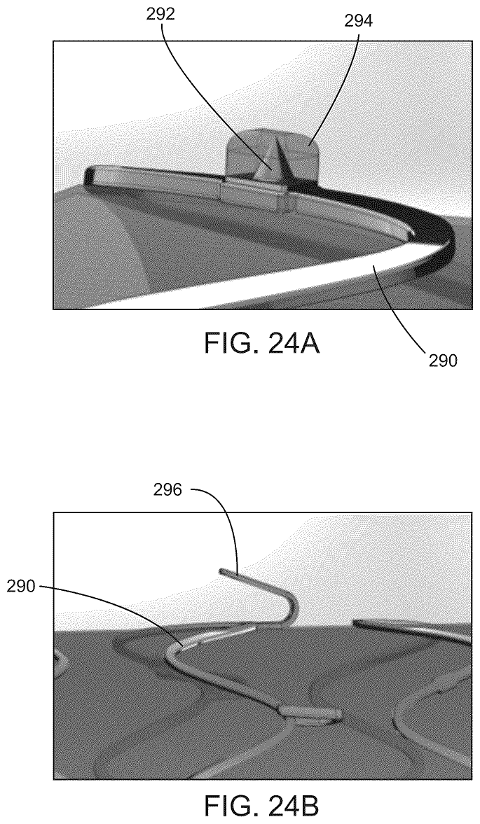

FIG. 24A illustrates a stent strut 290 having one or more penetrating surface electrodes 292 on the stent strut 290. These electrodes 292 penetrate the vessel wall after an encapsulating polymer structure 294 (e.g., crystalline glucose structure) is absorbed into the bloodstream over a time period. This structure allows the electrode 292 to gradually penetrate the vessel wall to achieve extravascular recording.

FIG. 24B illustrates a surface-mounted, flexible tine feature 296, which carries one or more electrode surfaces. These tine features 296 are set on a stent structure 290. Inside the delivery catheter, the tine features 296 are flattened, then on deployment, as the stent body is unsheathed, the tines fold into their shape memory form (flexing through ˜150 deg), penetrating the vessel wall as they do so and providing extravascular recordings along with secure fixation.

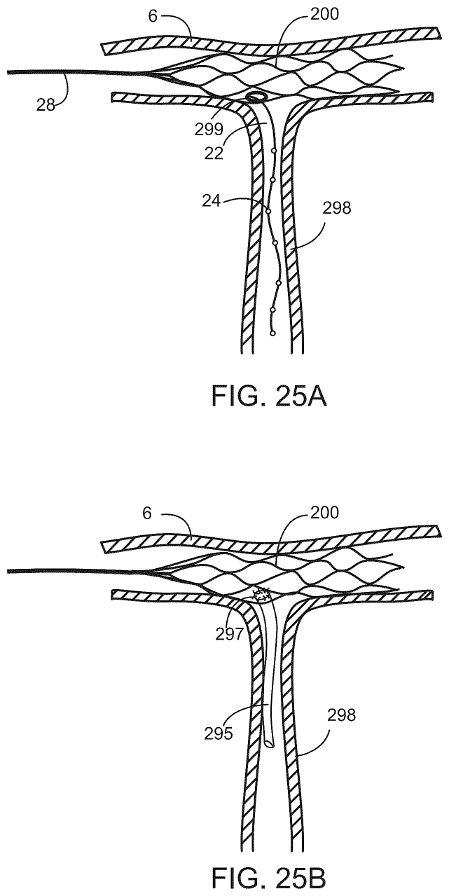

FIGS. 25A and 25B show variations of systems where sensing/recording electrodes are separated or spaced from an anchoring structure. FIG. 25A shows a stent structure 200 that functionally anchors within a vessel 6. It is noted that any type of anchoring structure (e.g., an expandable stent, a coil, etc.) is within the scope of this disclosure. As shown, the stent structure 200 can optionally include a lead component that communicates with a processing unit of the sensing device. FIG. 25A shows the anchoring stent 200 positioned within a vessel 6 that, is structurally robust to support the anchoring stent 200. In some cases, loci in the brain that are rich in information can be useful as input for BCI can be reached through blood vessels. However, in some cases, the blood vessels leading to information-rich loci have a small diameter (typically <4 mm) and are very thin and/or delicate. The nature of such blood vessels presents a hurdle to navigating devices delicate blood vessels, where the risk of rupture is significant as intravascular devices are pushed through them. This is especially where intravascular devices need to be maneuvered through difficult vascular pathways, such as small angles. Deploying recording devices in deep brain locations, where the typically small vessels are not sufficiently large to host an intravascular device. By way of illustration, typically, the distal transition zone (or a micro-to-macro transition) of an intravascular device is its most bulky portion since it contains all the conductive components of the recording component and the connection to a conductive lead. Even if the electrodes of the recording device can fit within a small blood vessel, the transition zone may not.

Therefore, as shown in FIG. 25A, the stent 200 can include an electrode carrier 22 containing one or more electrodes 24, where the electrode carrier 22 is tethered to the anchoring stent/structure 200, but the carrier 22 extends into a delicate vessel 298. While FIG. 25A shows a single electrode carrier 22. However, any number of carriers 22 can be used. Moreover, the electrode carrier 22 can extend distally to the stent structure 200 instead of into a branching vessel. In another variation, the electrode carrier 22 can extend through the vessel wall to contact brain tissue.

The carrier 22 can extend through a port 299 in the stent 200 or can extend through an opening. In one example, the stent 200 is deployed in a large vessel, such as the superior sagittal sinus (SSS), and serves as the port or foundation for the delivery (and supporting interface) of the electrode carrier 22, which is the recording portion that can be deployed in a branching cortical vein. The stent 200 can include any number of orientation features to assist in aligning the stent and/or electrode carrier.

In some variations, the electrode carrier can extend perpendicular to a longitudinal axis of the stent 200 through the port 299 in the stent 200.

FIG. 25B illustrates another variation of a device that separates a recording/sensing component from an anchoring structure. In this variation, an anchoring stent 200 positioned within a structurally sound vessel 6 anchors a recording component 295, which can include a stent or other electrode carrier. The variation shown in FIG. 25B shows an anchoring feature within a vessel having any number of anchors 297 (e.g., Nitinol anchors) that fix the stent structure 200 with the recording component 295. As noted above, this anchoring technique can mitigate risks of anchoring directly to delicate walls of small branch vessels 298, whose walls can be very delicate. Since the stent 200 is anchored into durable tissue, such as a venous sinus, the recording component 295 does not need to engage delicate walls of the target vessel 298. This technique has the added benefit that it supports a broader range of conductor wires or micro-to-macro transition zones than would be supported by the branch vessel alone, allowing a longer or thicker transition. Applied somewhere other than a branch, it could be the foundation to a device heading into deeper brain tissue.

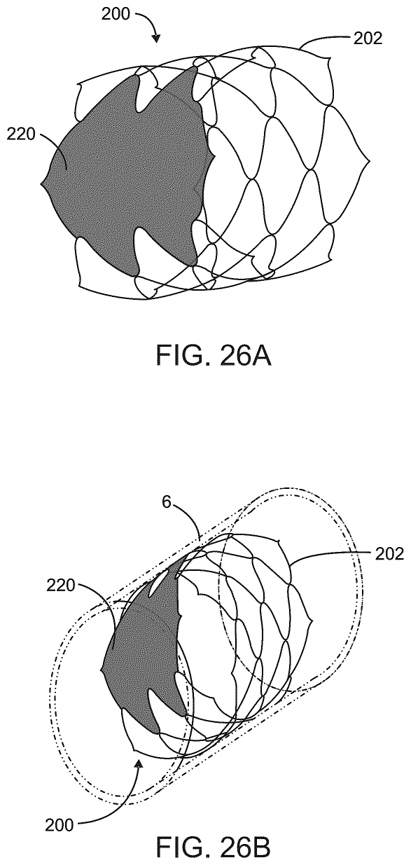

FIGS. 26A and 26B show an example of a self-expanding or balloon expanded stent 200 where a stent body 202 includes a frame with large cell size with a region of thin, soft, self-healing, polymer material 220 over an opening in the stent body 202. The polymer material 220 can be a mesh or single layer polymer sheet that is designed to be pulled taught by the expanding stent and produce a parallel plane arrangement with a wall of the vessel 6. The polymer material 220 can be configured to provide mechanical support for the puncture and deployment system and provides an improved barrier seal from blood/CSF flow across the puncture site.

This stent can be delivered to the target location within the blood vessel via combination of guide and delivery catheters. The operator takes care to orient the stent so that the polymer region is aligned with the portion of the vessel that is targeted for puncture. The operator then deploys the stent in the vessel using a puncture system (such as described above) to puncture through the polymer region of the stent device.

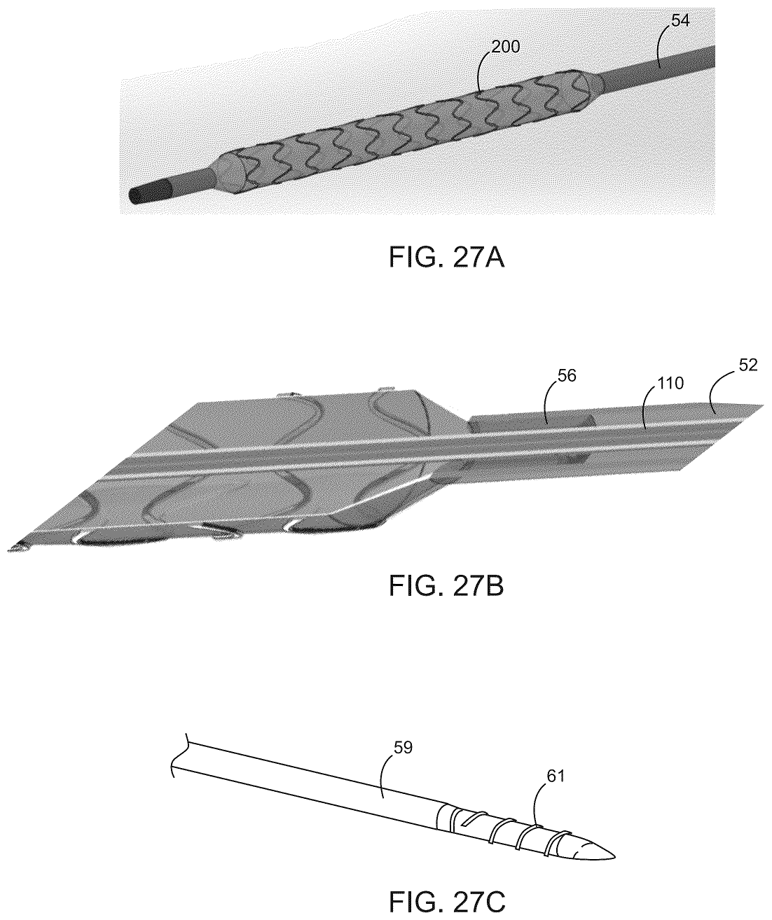

FIG. 27A illustrates a variation of a guide catheter 54 with an expanding stent-type feature that provides positional fixation within a target vessel. Expansion of the stent structure 200 can be achieved by removal of an internal dilation catheter, which elongates the distal portion, reducing the stent O.D. Upon arrival in a target vessel, the dilator is removed, allowing the distal guide catheter to foreshorten and expand radially, which also opens the internal lumen. As shown in FIG. 27B, which is a partial cross-sectional view of the device of FIG. 27A, the internal lumen 52 of the catheter 54 can also include a lumen-reducing balloon 56 or ratchet style feature. A piercing assembly 110 can be passed through the internal lumen 52 and angled as required for vessel puncture. When alignment is confirmed, the internal balloon 56 within the catheter 54 can be inflated to lock the piercing assembly 110 in position. Alternatively, or in combination, ratchet features can engage with the piercing assembly 110, locking its position relative to the already fixed guide catheter. This alignment may be further supported by a cradle system at the proximal end of the catheter systems and the use of a stereotactic frame system that allows precise understanding of relative positioning of all the catheters in use.

FIG. 27C illustrates another variation of a deep brain stimulation burrowing guidewire 59 that can optionally have a distal threaded portion 61 that can be advanced into brain tissue and achieve penetration via rotation. As shown, the guidewire 59 can include an extruded spiral thread portion 61 on the distal portion that allows the guidewire to be rotated by the operator to achieve a burrowing of the tip into tissue. This guidewire 59 can be used in instances where tracking through tissue from a blood vessel to a deep-brain structure is required. The guidewire 59 can include an outer catheter portion (not shown) that can be left in situ when the wire 59 is withdrawn to leave an open lumen through which any secondary device or system can be advanced. The guidewire may also use an electrode for electrical signal recording, cauterization, or other E.P. applications.

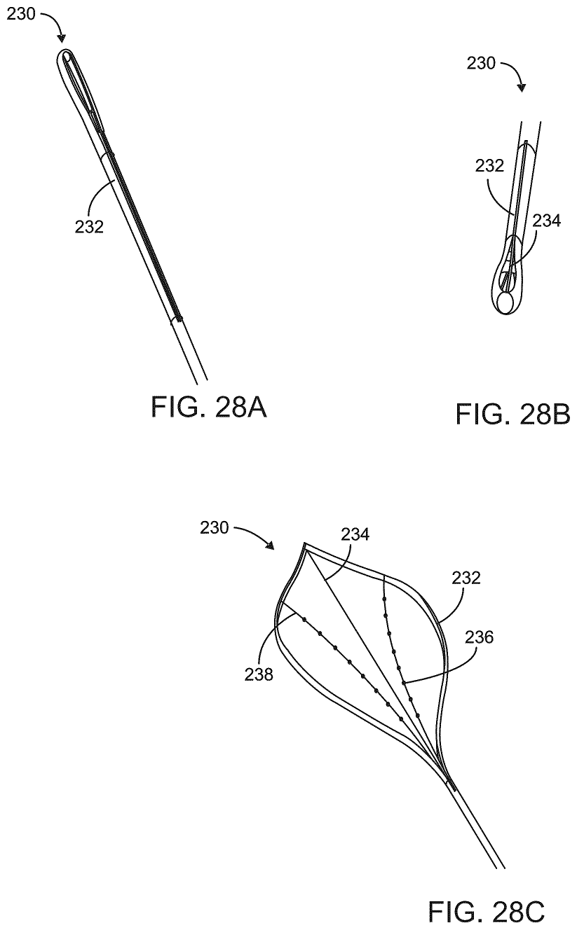

FIGS. 28A to 28C illustrate a variation of an electrode carrier 230 for deployment through the vessel wall. FIG. 28A shows a partial perspective view of the electrode carrier 230 in a delivery configuration where the electrodes are contained between adjacent arms 232. FIG. 28B shows a view of the electrode carrier 230 from a front of the carrier 230, showing a pull wire 234 located between adjacent arms 232. The arms 232 allow the electrode carrier 230 to have an atraumatic shape for navigation to the vessel site using a catheter system.

The low cross-sectional area/crossing profile that is achieved in the collapsed state allows the device to be delivered transvascular with a mitigated risk of gross vessel damage. As shown in FIG. 28C, the pull wire 234 can retract to expand the arms 232 to expose one or more splines 238 carrying one or more electrodes 236. In some variations, the splines 238 are configured to be electrodes. The ability to expand the electrode carrier in a planar direction allows the electrode array to monitor of a larger area with any number of electrodes.

To change the device state into a 2-dimensional recording/stimulation array, a proximal feature on the lead is actuated by the operator. This retracts the distal tip of the device, causing a slit section of the lead to separate and bow outwards. Several rib features that are connected to the inner surface of the lead outer jacket and nested within the collapsed lead then splay out as the bowing occurs. Each of the rib features contains a multitude of electrodes, which are spread in a 2-dimensional spatial array across the brain surface. Additional features, such as small mesh pads may be incorporated into the design to promote targeted endothelialization as a method of securing the array in place.

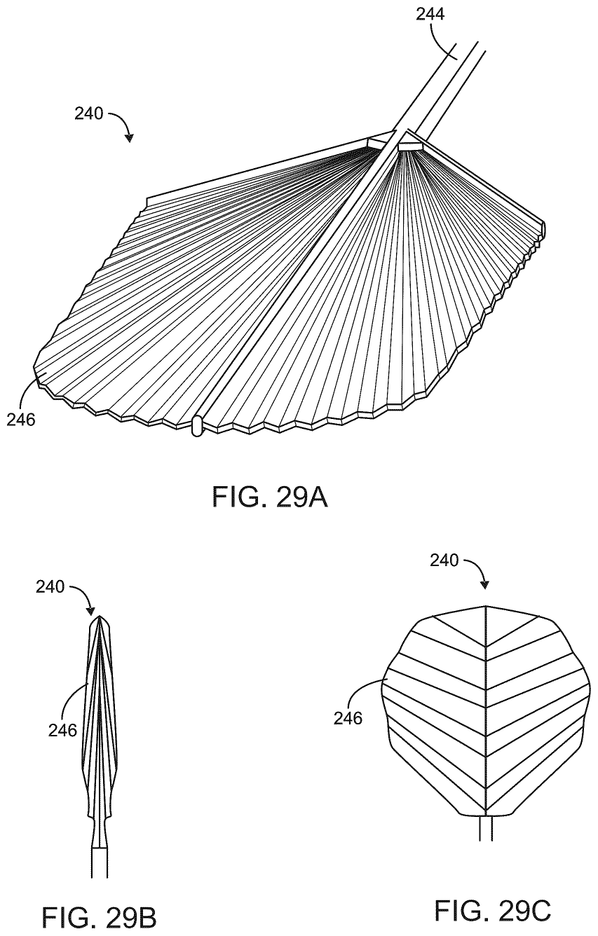

FIGS. 29A to 29C illustrate a variation of an electrode carrier 240 having a folding array 246 containing any number of electrodes where the array is located at the end of a catheter shaft 244. The interchanges between two deployment states are shown in FIGS. 29B and 29C. The collapsed state allows the electrode carrier 240 to be delivered through catheters/vascular system to the target site), and then expanded (for end recording use). The collapsed state, shown in FIG. 29B, produces a conformal array with the minimum crossing profile of the array to allow safe passage through small vessels and catheter systems. The expanded planar state, shown in FIG. 29C, produces a flexible, 2D array with the ability to arrange recording/stimulation electrodes spatially across the surface to achieve a maximized recording area. Expansion of the array can be achieved through various methods including operator actuation using cable features, self-expanding rib/spine features, thermally actuating polymers, fluid absorption actuation polymers, etc.

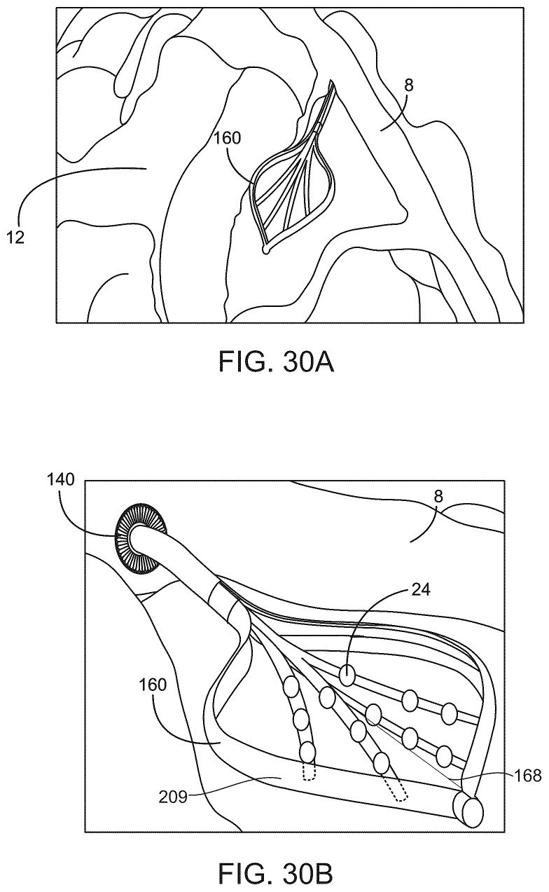

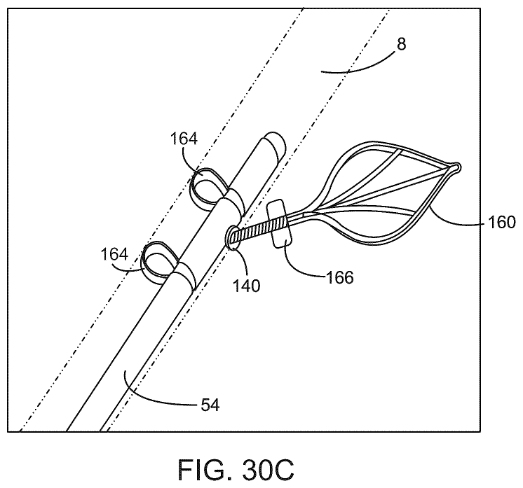

FIGS. 30A to 30C illustrate another variation of an expandable electrode carrier 160 extending out of a superior sagittal sinus 8 for positioning over brain tissue 12. FIG. 30A illustrates the electrode carrier 160 in a deployed state for recording neural signals. As noted above, the collapsed state of the carrier 160 provides a conformal array with the minimum crossing profile to permit safe passage through small vessels and other catheter systems. The expanded state shown in FIGS. 30A and 30B produce a flexible, 2D array with recording/stimulation electrodes 24 arranged spatially across the surface to achieve a maximized recording area. Expansion of the array can be achieved through various methods including operator actuation using cable features, self-expanding rib/spine features, thermally actuating polymers, fluid absorption actuation polymers, etc. As shown, the electrode carrier 160 can advance through the superior sagittal sinus (SSS) 8 or other vessels through a fixation device or grommet 140, which can also act as an anchor.

In some embodiments, grommet 140 is incorporated onto the lead body of an implanted neural interface device. On delivery to the target site, the operator can retract a feature at the proximal lead end (threaded, pull, etc.) that retracts an inner wire 168 affixed to the distal tip of the lead or the grommet 140. This process retracts the distal tip, resulting in the dissecting arms 209 of the grommet 140 separating to dissect tissue and also expose an N electrode array that fans out. The end result is an array with N electrode supporting elements that is spread in a 2D fashion over a target brain region. The device can also be retracted by reversing the process and withdrawing the contained lead body back through its delivery system.

FIG. 30C illustrates a guide catheter 54 within a SSS 8, where the catheter includes expanding features 164 to secure a side port against a wall of the SSS vessel 8. In this variation, the electrode carrier 160 extends through the vessel wall and through a self-expanding puncture port similar to the grommets described here. In addition, the electrode carrier 160 can include one or more fixation tabs 166 that permits endothelization to the dura or cortical surface to secure the electrode carrier 160 to a desired location.

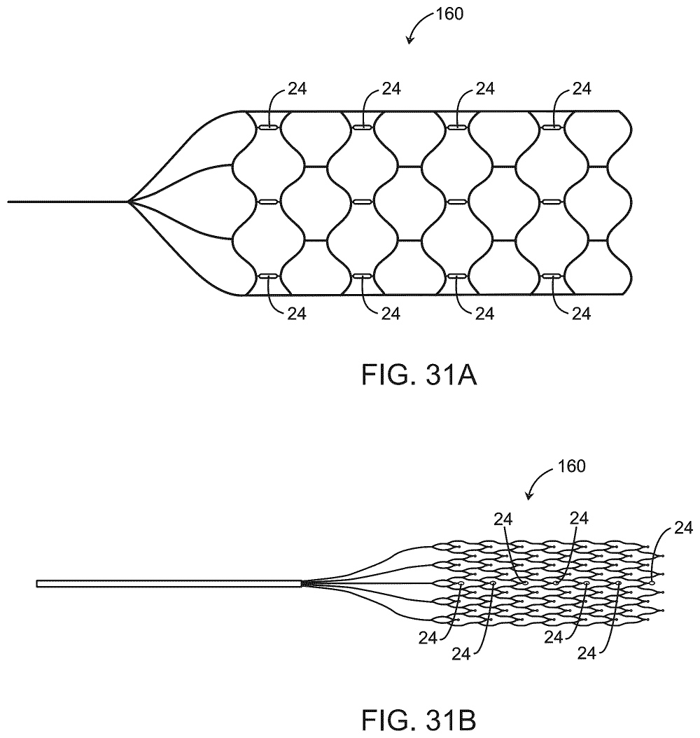

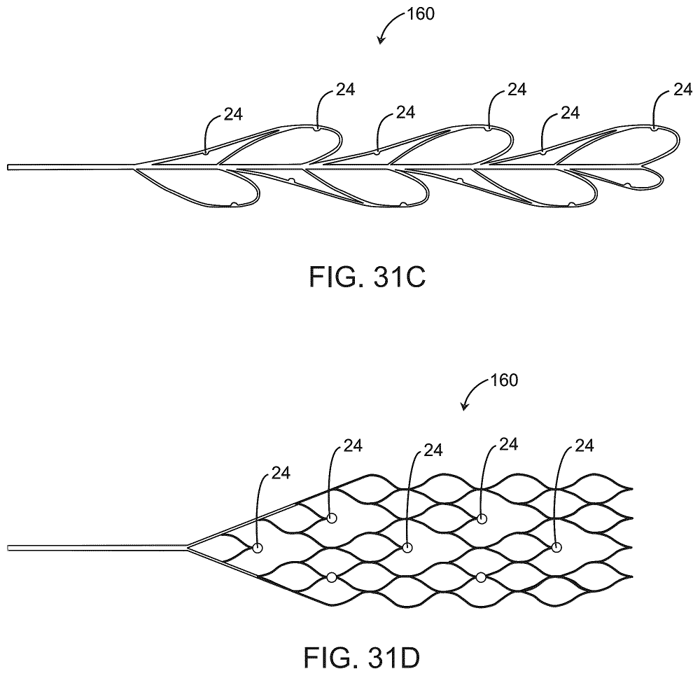

FIGS. 31A to 31D illustrate additional variations of electrode carriers 160 that carry various electrodes 24, where such devices provide stent electrode arrays that can be planar or flexible in shape. Such examples are similar to the tubular designs shown in the applications and patents referenced below. However, the electrode carriers 160 can be configured to have a flexibility, allowing for the electrodes 24 to cover more brain tissue as needed. For example, such planar configurations can conform to regions of the brain (e.g., the subarachnoid space).

Additional examples of tubular structure that can be reconfigured into planar electrode carriers can be found in the following patents and provisional applications 10,575,783; 10,485,968; 10,729,530; 63/370,164; 63/517,495 and 63/370,169. The entirety of each of which is incorporated by reference.

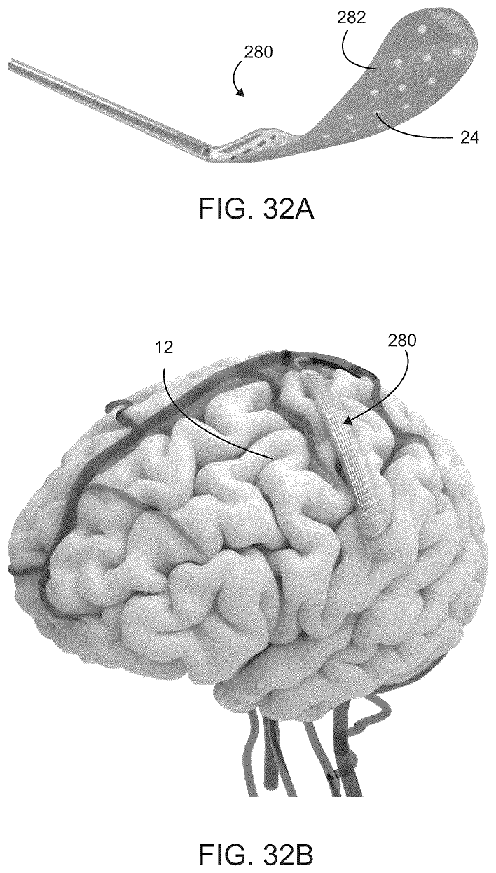

FIG. 32A shows a flexible microarray 280 comprising a polymer/ceramic substrate 282 that is highly flexible and capable of being rolled/folded/drawn into a delivery catheter. The substrate 282 carries a number of electrodes 24. The catheter can be tracked to the target deployment location where the array is unsheathed and “draped” over the cortical surface of the brain 12 as shown in FIG. 32B. The substrate 282 can comprise a hydro-channel array that is a flexible, folding electrode array actuated for final placement on a cortical target of the brain 12 by the injection (via a balloon indeflator or similar) of saline into a channel that runs through and around the array. The increase in pressure within this channel, as applied by the operator, causes the channel to expand, resulting in a controlled “unfolding” of the array at the target location. This mechanism can be useful in array substrates that are not capable of self-expanding or to eliminate the need for other metallic mechanical features. The electrode array may contain chain-mail articulating components that enable it to conform to the surface of the brain. In another configuration it has a rigid polymer layer, such as polyamide, that enables it to ‘roll-up’ to be inserted in a catheter, but also to ‘roll-out’ when delivered out of the catheter. To improve the durability of the device, a second layer of polymer with long-term biocompatibility, such as silicone or thermoplastic urethane (TPU) can be used to provide long-term biocompatibility. The device can be recaptured by retracting and rolling back up into a catheter for removal. In additional variations, the device may comprise a nitinol spine or skeleton to provide self-expanding properties

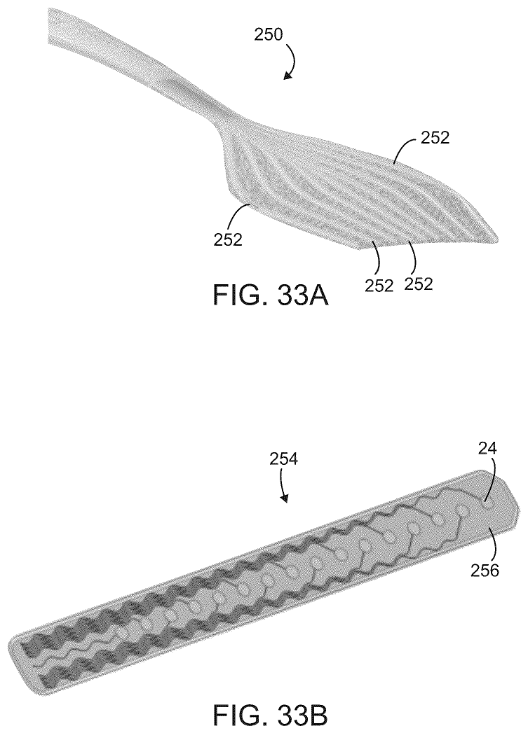

FIG. 33A shows a shape set polymer array 250 with a series of linear electrode arms 252. This array 250 can be unsheathed at a target and can passively/actively expand and spread over a wide cortical area. The high degree of flexibility in each thin arm results in an array that is less likely to cause any trauma or injury to delicate anatomical structures within the area. FIG. 33B shows an atraumatic array 254 with electrodes 24 produced by first constructing a hydrogel paddle 256 impregnated with a fine mesh, which acts to constrain the volume of the paddle during hydration/dehydration. The paddle 256 is then dehydrated, resulting in a polymer substrate onto which electrical traces and electrodes can be deposited. The paddle is then re-hydrated to produce a highly flexible electrode array that will conform to the cortical surface and provide an atraumatic array. Any of the implanted devices described herein can comprise different materials to balance ease of delivery with long term biocompatibility. For instance, the implants can comprise a polyamide layer, which assists in delivery and/or deployment of the implant, with a second layer, such as silicone or TPU to provide durability/long-term biocompatibility. The device can be recaptured. In some variations, silicone or TPU can have surface treatments to enhance lubricity or reduce the tack of the silicone surface. In some variations, a polymer can include fibers such as glass fibers, microfibers, PTFE fibers that are embedded in the tacky polymers, where the fibers provide actual structural integrity strength and reduce surface friction. In an additional variation, the device may contain a hermetically encapsulated chip that enables the multiplexing of one channel to many electrodes. The hermetic encapsulation allows the chip to remain housed inside the blood vessel while the distal portion of the device (electrode array) sits on the other side of the blood vessel wall on the surface of the brain.

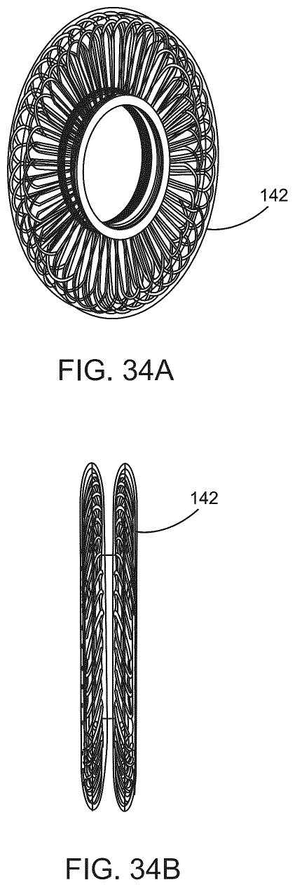

FIGS. 34A and 34B illustrate perspective and side views respectively, of an example of an expanding vessel puncture port or grommet 142.

It is noted that the concepts above while being illustrated as separate applications, can be combined in whole or in part. Features that are described in the context of separate aspects and embodiments of the invention can be used together and/or be interchangeable. Similarly, features described in the context of a single embodiment may also be provided separately or in any suitable combination.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

We claim:

1. A device for positioning one or more electrodes at a brain tissue, the device comprising:
a delivery catheter comprising a lumen;
a piercing assembly extending through the delivery catheter, wherein the piercing assembly comprises a piercing assembly catheter and a needle;
one or more electrodes configured to contact the brain tissue; and
wherein the piercing assembly catheter comprises an opening in a wall thereof such that when the delivery catheter and the piercing assembly catheter are positioned within a vessel, the needle extends through the opening to puncture a vessel wall.

2. The device of claim 1, wherein the one or more electrodes are configured to measure to measure progress of the one or more electrodes against or through the brain tissue.

3. The device of claim 1, wherein the one or more electrodes are positioned on the needle.

4. The device of claim 1, wherein the one or more electrodes are positioned on an electrode delivery device.

5. The device of claim 1, further comprising a biasing element coupled to the piercing assembly catheter, wherein the biasing element is configured to provide a counter force when the needle penetrates the vessel wall.

6. The device of claim 1, further comprising a locking structure on the delivery catheter, the locking structure configured to provide an interference surface between the delivery catheter and the piercing assembly catheter.

7. The device of claim 1, wherein the piercing assembly catheter comprises a helical shape.

8. The device of claim 7, wherein the piercing assembly catheter is configured to be rotated to control puncture of the vessel wall in a desired direction.

9. The device of claim 1, further comprising a balloon catheter configured to be advanced through the delivery catheter, wherein the balloon catheter comprises a balloon configured to expand to create a cavity between the brain tissue and a dura.

10. The device of claim 9, further comprising a second balloon configured to be delivered by the balloon catheter.

11. The device of claim 9, further comprising one or more electrodes positioned on the balloon catheter, wherein the one or more electrodes are configured to determine whether the cavity is created.

12. The device of claim 9, wherein the balloon is configured to transition from a rolled configuration to an unrolled configuration.

13. The device of claim 1, further comprising a jaw device configured to be advanced through the delivery catheter, wherein the jaw device is configured to dissect a dural tissue to create a cavity between the brain tissue and a dura.

14. The device of claim 13, wherein the jaw device is configured to measure impedance to determine whether the cavity is created.

15. The device of claim 1, further comprising a wedge device configured to be advanced through the delivery catheter, wherein the wedge device is configured to dissect a dural tissue to create a cavity between the brain tissue and a dura.

16. The device of claim 1, further comprising one or more pull wires configured to steer the delivery catheter.

17. The device of claim 1, further comprising a port located on or through an opening in a skull and external to the brain tissue, wherein a positioning catheter is passed through the port and is coupled to the one or more electrodes to place the one or more electrodes.

18. The device of claim 1, further comprising a rotational body within the delivery catheter, wherein the rotational body is fixed to the delivery catheter at a distal end of the rotational body and is configured to rotate the delivery catheter upon a proximal force applied to the rotational body.

19. A method of positioning one or more electrodes over brain tissue, the method comprising:
delivering a delivery catheter into a vessel lumen;
puncturing a vessel wall at a target site with a piercing assembly extending from the delivery catheter, wherein the piercing assembly comprises a needle;
forming a cavity between the brain tissue and a dura, wherein the cavity provides a space adjacent to the brain tissue and the dura; and
positioning the one or more electrodes in the space such that the one or more electrodes can record neural activity.

20. A method of advancing one or more electrodes to a desired target, the method comprising:
delivering a delivery catheter into a vessel lumen of a vessel;
advancing an anchor structure through the delivery catheter and within the vessel lumen at a desired location within the vessel;
advancing a navigation device through the delivery catheter;
positioning the navigation device outside the vessel, wherein the navigation device is configured to be articulated to align a travel path with the desired target; and
advancing an electrode carrier through the navigation device, wherein the electrode carrier comprises one or more electrodes and is advanced along the travel path until the one or more electrodes are positioned at the desired target.
